(19) United States
(12) Patent Application Publication
(10) Pub. No.: US 2023/0151334 A1
(43) Pub. Date: May 18, 2023

(54) CULTURE PLATFORMS, METHODS, AND USES THEREOF

(71) Applicants: HELSINGIN YLIOPISTO, Helsingin Yliopisto (FI); AALTO UNIVERSITY FOUNDATION SR, Aalto (FI)

(72) Inventors: Pauliina MUNNE, Helsinki (FI); Juha KLEFSTRÖM, Helsinki (FI); Lahja MARTIKAINEN, Aalto (FI); Nonappa NONAPPA, Aalto (FI); Kia BERTULA, Aalto (FI); Olli IKKALA, Aalto (FI)

(21) Appl. No.: 17/916,434

(22) PCT Filed: Apr. 1, 2021
(86) PCT No.: PCT/FI2021/050241

§ 371 Date: Sep. 30, 2022

(30) Foreign Application Priority Data
Apr. 2, 2020 (FI) 20205335

(51) Int. Cl. C12N 5/09; C12M 1/12; G01N 33/50

(52) U.S. Cl. CPC C12N 5/0693; C12M 25/14; G01N 33/5044; C12N 2500/02; C12N 2501/727; C12N 2513/00; C12N 2527/00; C12N 2533/74

(57) ABSTRACT

The present invention relates to the fields of life sciences and cell and tissue cultures, especially 3D cultures. Specifically, the invention relates to a method of maintaining the presence or activity of a human or mouse estrogen receptor (ER) in a cell of an ex vivo mammary cell or tissue culture or in a cell of other hormone responsive cell or tissue culture. Also, the present invention relates to a method of maintaining a luminal epithelial phenotype and/or cell identity of a mammalian cell in an ex vivo cell or tissue culture. Still, the present invention relates to a 3D matrix or 3D medium comprising the matrix for ex vivo culture, wherein said 3D matrix or 3D medium comprises one or more mammalian cells or tissues embedded in said 3D matrix or 3D medium, and to a system for ex vivo culture, wherein the system comprises mammalian cells or tissues embedded in a 3D matrix or 3D medium comprising said matrix. Still furthermore, the present invention relates to use of the 3D matrix, 3D medium or system of the present invention e.g. for ex vivo culture of a mammalian cell, drug discovery methods, biomarker studies and/or estrogen receptor (ER) signaling studies.

### FIELD OF THE INVENTION

The present invention relates to the fields of life sciences and cell and tissue cultures, especially 3D cultures. Specifically, the invention relates to a method of maintaining the presence or activity of a human or mouse estrogen receptor (ER) in a cell of an ex vivo mammary cell or tissue culture or in a cell of other hormone responsive cell or tissue culture. Also, the present invention relates to a method of maintaining a luminal epithelial phenotype and/or cell identity of a mammalian cell in an ex vivo cell or tissue culture. Still, the present invention relates to a 3D matrix or 3D medium comprising the matrix for ex vivo culture, wherein said 3D matrix or 3D medium comprises one or more mammalian cells or tissues embedded in said 3D matrix or 3D medium, and to a system for ex vivo culture, wherein the system comprises mammalian cells or tissues embedded in a 3D matrix or 3D medium comprising said matrix. Still, furthermore, the present invention relates to use of the 3D matrix, 3D medium or system of the present invention e.g. for ex vivo culture of a mammalian cell, drug discovery methods, biomarker studies and/or estrogen receptor (ER) signaling studies.

### BACKGROUND OF THE INVENTION

Some cancers, e.g., including breast, prostate, and ovarian cancers are variable in terms of patient outcomes and relapse can occur eventually in a large number of patients. Therefore, new therapeutics and approaches for better drug targeting to the right patient groups need to be developed.

The tremendous failure rate of new potential cancer drugs in the clinical trials has become a bottleneck in the drug development. The vast majority of new therapies fail in the late-phase clinical trials, which has resulted in highly inefficient and costly oncology drug development processes. Poor predictivity of the preclinical models due to the failure to recapitulate the proper tumor biology has become one of the major reasons for this.

The traditional cell culture conditions or protocols strongly promote either differentiation or lead to wrong cell types from the initially heterogeneous populations, due to currently unknown reasons. Cancer cell lines have been proven to be a valuable source of information for drug discovery process, but their limitations have been increasingly recognized.

With the current technology, the patient-derived xenograft (PDX) process is laborious and expensive, thus limiting the number of drugs and combinations that can be tested. For these reasons, there has been an increasing need to develop new more tailored PDX models by implanting malignant tissues in three-dimensional (3D) culture systems.

Biomimetic ex vivo culture systems from patient-derived tissues hold promise to revolutionize concepts of personalized treatment of solid tumors, providing attractive alternatives for reductionist and artifact-prone cancer cell lines and long passaged primary tumors. Therein, tumor explants provide simultaneously a source of patient-specific molecular information and live tumor samples for testing novel treatment options with respect to the obtained molecular information. In the past several years, a tremendous effort has been put into development of 3D culture systems and adopting them in drug discovery, cancer cell biology, and stem cell studies. The biggest challenges of 3D models are to maintain the viability of tumor samples in long-term cultures and to prevent them from changing the cellular identity and the overall cellular heterogeneity of the original tumor during culture period. Indeed, it has been challenging to establish 3D cell or tissue culture systems due to difficulties to reconstitute optimized biochemical and physical microenvironment for tissues.

For example, up to 80% of breast cancers are of the luminal estrogen receptor positive (ERα+) subtype and the development of ERα pathway-targeted medications has been one of the greatest advances in oncology. However, despite the need for novel ERα pathway targeting drugs, there are only few ERα+ preclinical models available for the drug discovery, development and testing. In the prior art, the existing ex vivo tumor tissue models show rapid loss of ERα expression in the culture conditions (Graham J. D. et al. 2009, Endocrinology 150, 3318-3326; Tanos T. et al. 2013, Sci Transl Med 5, 182ra155). In culture, the slowly growing ERα+ cells become outcompeted by the faster growing ERα- cell clones, or ERα is lost due to the incongruent culture conditions; either there is a lack of necessary growth factors or the growth medium contains growth factors with unwanted effects on the ERα epithelial phenotype (Petersen, O. W. and van Deurs, B. 1988, Differentiation 39, 197-215; Taylor-Papadimitriou, J. et al., 1989, J Cell Sci 94 (Pt 3), 403-413).

Therefore, tractable methods and 3D cell or tissue culture models are critically needed for preserving estrogen receptors, as well as for preserving also the luminal epithelial phenotype and/or cell identity of mammalian cells in an ex vivo cell or tissue culture.

### BRIEF DESCRIPTION OF THE INVENTION

Loss of the hormone reseptors and other phenotypic features of tumor samples has been a major caveat of different ex vivo culture models. Understanding how cells sense and respond to the biochemical and mechanical cues based on their surroundings is crucial for understanding the regulation of tumor cell identity. The prior art 3D matrices for cell and tissue culture strongly promote a loss of functional hormone receptor expression and in the switch in the cellular identity from luminal to basal, which are unwanted effects in several tumors, e.g., in most of breast cancer tissues, as the majority of breast cancers form estrogen receptor positive luminal tumors. Switches of cellular identities from luminal to basal or loosing the presence or activities of estrogen receptors can result e.g. from batch-to-batch variation of the prior art matrices, unknown growth factors or compositions of the prior art matrices and/or a lack of stability under culture conditions. The prior art methods lack an ability to tune specific mechanical properties of 3D matrices on demand.

The objects of the present invention, namely methods of maintaining the activity of estrogen receptors (ERs) and/or the presence of a luminal epithelial phenotype cell identity of a cell in a 3D medium or matrix, are achieved by utilizing a specific matrix for ex vivo culture, to be explained in this invention. The present invention suggests to overcome the defects of the prior art tissue culture models, including but not limited to problems in maintaining the functional ERα, poor viability of tumor cells or tissues in long-term 3D cultures, lack of ability to preserve the luminal epithelial phenotype and/or cellular identity and the overall heterogeneity of the original tissue or tumor during culture period.

It has here been found that when a mammalian cell or tissue such as a mammary or other hormone responsive cell or tissue is cultured in a 3D matrix or 3D medium comprising said matrix having the mechanical stiffness at least about 10 kPa, luminal properties or identity of said cell or tissue can be maintained. Even more importantly, specific stiffness of the matrix enables the creation of 3D media for mammalian cell cultures preserving e.g. the ERα+ luminal phenotype.

On the other hand it has also been found here that when a mammalian cell or tissue is cultured in a substantially gelled and/or bioinert 3D matrix or 3D medium comprising a substantially gelled and/or bioinert matrix, luminal properties or identity of said cell or tissue can be maintained.

The results of the present disclosure further reveal that mechanical and/or biochemical composition of a matrix, which mimics the microenvironment of the body, plays an important role in maintenance of tumor specific gene expression profiles. The microenvironment obtained by the methods and tools of the present invention is able to maintain appropriate gene expression profiles of the original cells or tissue samples and thus also the correct molecular mechanisms regulating their cell growth and cellular responses in vitro.

With the methods and matrix of the present invention epithelial cell identity can be controlled. An epithelial cell identity and the presence of ERα in a cell can be independently regulated. Also, the present invention enables maintaining the stability of basal or luminal epithelial cell identities. The present invention enables tuning of specific mechanical and/or biochemical properties of 3D matrices on demand and thereby allow maintaining ERα or epithelial cell identity of cells or tissues cultured in said matrices.

Further advantages of the present invention include but are not limited to a simple method for 3D ex vivo or in vitro culture and matrix with cost-effective materials. The platform of the present invention is very controllable, it allows high throughput studies, and only a short time is needed for achieving results with low costs.

The present invention relates to a method of maintaining the presence or activity of a mammalian estrogen receptor (ER) in a cell of an ex vivo mammary cell or tissue culture or in a cell of other hormone responsive cell or tissue culture, wherein the method comprises culturing a mammalian mammary cell or tissue or other hormone responsive cell or tissue in a 3D matrix or 3D medium comprising said matrix, wherein the mechanical stiffness (e.g. storage modulus (G′)) of the matrix is at least about 10 kPa, optionally measured by dynamic rheology.

The present invention relates to a method of maintaining the presence or activity of a human estrogen receptor (ERα) in a cell of an ex vivo mammary cell or tissue culture or in a cell of other hormone responsive cell or tissue culture, wherein the method comprises culturing a human mammary cell or tissue or other hormone responsive human cell or tissue in a 3D matrix or 3D medium comprising said matrix, wherein the mechanical stiffness (e.g. storage modulus) of the matrix is at least about 10 kPa, optionally measured by dynamic rheology.

Also, the present invention relates to a method of maintaining the presence or activity of a mouse estrogen receptor (ERα) in a cell of an ex vivo mammary cell or tissue culture or in a cell of other hormone responsive cell or tissue culture, wherein the method comprises culturing a mouse mammary cell or tissue or other hormone responsive mouse cell or tissue in a 3D matrix or 3D medium comprising said matrix, wherein the mechanical stiffness (e.g. storage modulus) of the matrix is at least about 10 kPa, optionally measured by dynamic rheology.

Also, the present invention relates to a method of maintaining a luminal epithelial phenotype and/or cell identity of a mammalian cell in an ex vivo cell or tissue culture, wherein the method comprises culturing a mammalian cell in a 3D matrix or 3D medium comprising said matrix, wherein the mechanical stiffness (e.g. storage modulus) of the matrix is at least about 10 kPa, optionally measured by dynamic rheology.

Also, the present invention relates to a 3D matrix or 3D medium comprising the matrix for ex vivo culture, wherein said 3D matrix or 3D medium comprises one or more mammalian cells or tissues embedded in said 3D matrix or 3D medium, wherein the mechanical stiffness (e.g. storage modulus) of the matrix is at least about 10 kPa, optionally measured by dynamic rheology.

Also, the present invention relates to a system for ex vivo culture, wherein the system comprises one or more mammalian cells or tissues embedded in a 3D matrix or 3D medium comprising said matrix, wherein the mechanical stiffness (e.g. storage modulus) of the matrix is at least about 10 kPa, optionally measured by dynamic rheology.

Still, the present invention relates to a method of culturing a cell or tissue (e.g. a mammalian, mouse or human cell or tissue) ex vivo, wherein the method comprises culturing a cell in the 3D matrix, 3D medium or system of the present invention in suitable conditions.

Still, the present invention relates to use of the 3D matrix, 3D medium or system of the present invention for ex vivo culture of a mammalian cell, drug discovery methods, biomarker studies and/or estrogen receptor (ER) signaling studies.

Still furthermore, the present invention relates to a method of maintaining a luminal phenotype (e.g. luminal epithelial phenotype) and/or luminal cell identity (e.g. luminal epithelial phenotype) of a mammalian cell in an ex vivo cell or tissue culture, wherein the method comprises culturing a mammalian cell in a 3D matrix or 3D medium comprising said matrix, wherein the matrix is substantially gelled, bioinert and/or the mechanical stiffness of the matrix is one or more selected from the group consisting of: at least about 0.1, 0.5, 1, 2, 3, 4, 5, 6, 7, 8, 9 or 10 Pa (e.g. at least about 20, 30, 40, 50, 60, 70, 80, 90 or at least about 100 Pa) optionally measured by dynamic rheology and presented as either storage modulus or elastic modulus; at least about 0.1, 0.5, 1, 2, 3, 4, 5, 6, 7, 8, 9 or 10 kPa optionally measured by dynamic rheology and presented as either storage modulus or elastic modulus.

Still furthermore, the present invention relates to a 3D matrix or 3D medium comprising the matrix for ex vivo culture, wherein said 3D matrix or 3D medium comprises one or more mammalian cells or tissues embedded in said 3D matrix or 3D medium, wherein the matrix is substantially gelled, bioinert and/or the mechanical stiffness of the matrix is one or more selected from the group consisting of: at least about 0.1, 0.5, 1, 2, 3, 4, 5, 6, 7, 8, 9 or 10 Pa (e.g. at least about 20, 30, 40, 50, 60, 70, 80, 90 or at least about 100 Pa) optionally measured by dynamic rheology and presented as either storage modulus or elastic modulus; at least about 0.1, 0.5, 1, 2, 3, 4, 5, 6, 7, 8, 9 or 10 kPa optionally measured by dynamic rheology and presented as either storage modulus or elastic modulus.

Still, the present invention relates to a system for ex vivo culture, wherein the system comprises one or more mammalian cells or tissues embedded in a 3D matrix or 3D medium comprising said matrix, wherein the matrix is substantially gelled, bioinert and/or the mechanical stiffness of the matrix is one or more selected from the group consisting of: at least about 0.1, 0.5, 1, 2, 3, 4, 5, 6, 7, 8, 9 or 10 Pa (e.g. at least about 20, 30, 40, 50, 60, 70, 80, 90 or at least about 100 Pa) optionally measured by dynamic rheology and presented as either storage modulus or elastic modulus; at least about 0.1, 0.5, 1, 2, 3, 4, 5, 6, 7, 8, 9 or 10 kPa optionally measured by dynamic rheology and presented as either storage modulus or elastic modulus.

Still further, the present invention relates to a method for discovering drugs and/or studying biomarkers or estrogen receptor (e.g. ERa) signaling, wherein the method comprises culturing a cell or tissue (e.g. a mammalian, mouse or human cell or tissue) ex vivo in the 3D matrix, 3D medium or system of the present invention in suitable conditions (optionally in the presence of an external agent such as a drug), optionally determining biomarkers of said cell or tissue, and thereby discovering drugs and/or studying biomarkers or ER (e.g. ERa) signaling.

Other objects, details and advantages of the present invention will become apparent from the following drawings, detailed description and examples.

### BRIEF DESCRIPTION OF THE DRAWINGS

FIGS. 1A - E. A) A schematic illustration of the patient-derived explant culture (PDEC) platform. The original sample is processed into smaller pieces using an enzymatic treatment. These pieces are called explants and they are embedded in 3D matrices. B) The table illustrates the proliferation, apoptosis, and hypoxia of PDEC-N (N = normal mammary epithelial tissue based on a reduction mammoplasty sample) and PDEC-BC (BC = breast cancer tissue) after seven days (7 d) of culture. C) Mutation profiling was performed with TruSeq Amplicon - cancer panel. Primary breast tumors from three patients (P9T, P13T, P15T) were cultured in 3D for 7 d and Tp53 mutation profiles were compared to the original tumor samples. D) Basal cell identity promoting matrices (BMx) and E) luminal cell identity preserving matrices (LMx) using normal mammary epithelial tissues from mouse (MMEC) and from human reduction mammoplasty (PDEC-N), cultured in seven different 3D matrices. The effect on the luminal-basal cell identity was observed with immunofluorescent staining by using cytokeratin 8 (CK8) as luminal and cytokeratin (CK14) as a basal cell type marker. Rheological measurements of the analyzed matrices: Frequency sweeps show the storage, G′, and loss, G″, moduli as a function of the oscillation frequency. Elastic modulus, E, is calculated from the complex modulus, G*, with Poisson’s ratio, ν, of 0.5, E=2(1 +ν)G*. For Ag ν=0.44 is used according to Brewin et al. (2015, Ann Biomed Eng 43, 2587-2596).

FIGS. 2A - C. A) PDEC-N and PDEC-BC cultured in a luminal identity preserving matrix (LMx) and in a basal promoting matrix (BMx). Explants are stained with a luminal marker (CK8) and a basal cell marker (CK14). B) Quantification of luminal/basal identity in explant cultures. All explant cultures were divided in five different groups based on expression of CK8 and CK14 in individual cells. The first group (1) contained explants which showed only staining of the luminal marker. The second group (2) consisted of explants, which were mainly of luminal phenotype (75% of CK8 positivity), but had also some basal staining (25% of CK14 positivity). Explants in the third (3) category were equally positive for both markers. In the fourth group (4) the majority of the staining was basal (75% of CK14 positivity), but explants had still some luminal marker staining (25% of CK8 positivity). In the last group (5) the explants were solely basal. Images show examples in each group. C) GSEA analysis shows the enriched of gene sets indicating basal cell identity in the BMx-grown MMECs. (HUPER_BREAST_BASAL_VS_LUMINAL_DN; HUPER_BREAST_BASAL_VS_LUMINAL_UP and PID_DELTA NP63_PATWAY). Scale bar 10 µm.

FIGS. 3A - D. A) Principal component analysis (PCA) shows a separate clustering between BMx-Mat, LMx-Ag and LMx-Al according to their transcriptome. B) The corresponding rheological data for the different matrices, where only LMx-Ag is able to keep the ERα function. C) GSEA analysis shows lack of ERα function related profiles in LMx-Al compared to the uncultured samples. Note the negative NES values. D) GSEA analysis shows the enrichment of the estrogen receptor alpha (ERα) function related profiles in LMx-Ag compared to LMx-Al. Note the positive NES values.

FIGS. 4A - D. A) Immunofluorescent staining of ERα in MMECs with the increasing polymer concentration. ERα appears in LMx-Ag with concentrations above 20 mg/mL, i.e., when the gel is sufficiently stiff. B) The corresponding rheological data using LMx-Ag at different concentrations leading to widely tunable stiffnesses. C) Western blot analysis shows upregulation of ERα in triple negative breast cancer (TNBC) cell line with anisomycin treatment. MCF7 and T47D are ERα positive cell lines and serves as positive control. TGFβ treatment serves as a negative control. D) Immunofluorescent staining reveals activation of phosphorylated-38 together with nuclear ERα in PDEC-N and PDEC-BC after exposing them to anisomycin. Immunofluorescent staining reveals activation of nuclear ERα in MMECs, TNBC cell line DU4475, PDEC-N and PDEC-BC after exposing them to enhacer of zeste 2 (EZH2) inhibitor (GSK-126).

FIGS. 5A - E. A) Air-Liquid Interface (ALI) cultures activate p38p and preserves nuclear ERα expression in PDEC-N and PDEC-BC. B) The QRT-PCR results from ALI cultured PDEC-BC reveals downregulation of the progesterone receptor (PGR gene, PR protein) expression after o/n treatment with tamoxifen. C) Illustration of the permanent magnet mediated compression method. D) Expression of p38p and ERα in LMx-Ag cultured PDEC-N and PDEC-BC after overnight compression with the permanent magnets. E) Tamoxifen suppresses the expression of ERα target genes PGR in PDEC-BC cultures exposed to magnet-mediated compression. Analysis by QRT-PCR. Scale bar, 10 µm.

FIG. 6 reveals the collected G′, G″, G* and E of the studied matrices (average values +/- standard deviation) measured by dynamic rheology.

### DETAILED DESCRIPTION OF THE INVENTION

The results of the present disclosure from ex vivo 3D culture models reveal a maintenance of ER (e.g. ERα), luminal (epithelial) phenotype and/or cell identity in specific matrices. Indeed, it has been a long-standing grand challenge to preserve Erα expression in 3D cultured mammary tumor cells. Now, the culture matrices of the present invention or used in the present invention are able to resemble the microenvironment of the cells or tissues in vivo.

The method of the present invention concerns maintaining the presence or activity of an estrogen receptor (e.g. ERa) in a cell of an ex vivo mammary cell or tissue culture or in a cell of other hormone responsive cell or tissue culture. In one embodiment of the present invention the 3D matrix is capable of maintaining or preserving the presence or activity of an estrogen receptor (e.g. ERa) in a cell or tissue embedded in said matrix.

ERαs are expressed and present in many normal or tumor cells in vivo and maintaining the presence or activity of ERα ex vivo enables effective and reliable ex vivo cultures and studies on cells or tissues of said cultures. As used herein “an estrogen receptor (ER)” refers to a receptor, which belongs to the steroid hormone superfam-ily of nuclear receptors and can be activated by the hormone estrogen (17β-estradiol). After estrogen has activated the ER, said ER can translocate into the nucleus and for example bind to DNA thereby regulating the activity of different genes. For example, “activity of an ER or ERα” can refer to a genomic activity of an ER or ERα, respectively. As used herein “genomic activity of an ERα” refers to an ability of the ERα to bind DNA or to activate downstream target genes such as PGR, GREB1 or known ERα regulated gene sets. Two classes of ER exist: nuclear estrogen receptors Erα and Erβ. The estrogen receptor maintained or preserved by the method or matrix of the present invention can be any mammal estrogen receptor and can be selected from the group including but not limited to human, rodent, mouse, murine, hamster, rabbit, swine, dog, and cat estrogen receptors. ER refers to any ER homologue from any mammal, e.g., a human or mouse. Also, all isozymes, isoforms, and variants are included with the scope of ER.

The presence, absence, or amount of the ER or an activity thereof in a cell or tissue can be detected or measured by any suitable method known in the art. In one embodiment the method comprises determining the presence, absence, or amount of the ERα or an activity thereof. Determinations, detections or measurements suitable for the present invention can either directly or indirectly reveal the ER or activity thereof. For example, ER polypeptide expression, expression of the polynucleotide encoding ER (e.g., mRNA) or the presence of ER polypeptide can be utilized for stydying the presence or activity of an ER. Non-limiting examples of suitable detection methods include commercial kits on the market, enzymatic assays, immunological detection methods (e.g., antibodies specific for ER polypeptides), nucleic acid based methods, staining methods (e.g., immunofluorescent staining), and any combination thereof. For example, RNA and/or DNA -based methods are suitable nucleic acid methods for the present invention and include but are not limited to hybridization methods (e.g., southern or northern blotting, slot/dot blot, colony blot, fluorescence in situ hybridization, microarray), PCR methods (e.g., qPCR, RT-PCR, methylation-specific PCR, multiplex-PCR), and sequencing methods (e.g. basic sequencing methods, large-scale sequencing, high-throughput methods). In a specific embodiment the activity of the polypeptide is determined by monitoring expression of the polynucleotides regulated by the ER (e.g., PGR, GREB1, PCP4 and/or pS2).

In the present disclosure, the terms “polypeptide” and “protein” are used interchangeably to refer to polymers of amino acids of any length. Furthermore, as used herein “a polynucleotide” refers to any polynucleotide, such as single or doublestranded DNA (genomic DNA or cDNA) or RNA, comprising a nucleic acid sequence encoding a polypeptide in question or a conservative sequence variant thereof. Conservative nucleotide sequence variants (i.e. nucleotide sequence modifications, which do not significantly alter biological properties of the encoded polypeptide) include variants arising from the degeneration of the genetic code and from silent mutations.

As used herein “maintaining” or “preserving” the presence or activity of an ER (e.g. ERa) refers to the ability of the method or matrix of the present invention to continue an expression of the ER or any polynucleotide encoding ER, or to keep or produce an activity of an ER in a cell or tissue. “Maintaining” or “preserving” does not necessarily mean only maintaining or preserving 100% of the presence or activity of ERs (e.g. ERαs) in an ex vivo cell, cells or tissue compared to the presence or activity of an ER or Ers in an in vivo cell, cells or tissue, but also maintaining or preserving at least some, e.g. at least 10%, 20%, 30%, 40%, 50%, 60%, 70%, 80%, 90% or 95% of the Ers in a cell or tissue, or an activity of an ER or Ers in a cell or tissue, or at least 10%, 20%, 30%, 40%, 50%, 60%, 70%, 80%, 90% or 95% of the cells or tissue having Ers or active Ers. Depending on the studies carried on ex vivo cell or tissue cultures in 3D matrices or 3D media said cultures can be needed or last for example few hours to months. In one embodiment the presence or activity of an ER or ERs is maintained for at least two, three, four, five, six, seven, eight, nine or ten days (e.g. at least one, two, thee or four weeks) in an ex vivo culture.

It is very difficult to preserve ERs of normal and tumor cells or tissues in their cultures, including but not limited to mammary, prostate, ovary, kidney, liver, bladder, pancreas or cartilage cells or tissues. Particularly the ERs are commonly known to disappear from the 3D ex vivo cultures of breast, prostate, and ovarian cancer samples. Therefore, maintaining ERs in mammary cells and tissues as well as in other hormone responsive cells or tissues is of high interest. As used herein “other hormone responsive cell or tissue” refers to a cell of the prostate, chondrocyte, kidney cell, liver, pancreatic, bladder or ovarian cell, or a tissue comprising cells selected from the group consisting of cells of the prostate, chondrocytes, kidney cells, liver, pancreatic cells, bladder cells and ovarian cells, and any combination thereof.

In one embodiment of the method of maintaining the presence or activity of ER, the culture is exposed to an external stress or compound. External stress or compounds include but are not limited to, e.g., anisomycin or any other p38 activating compound and an EZH2 inhibitor (e.g., GSK-126). In one embodiment the cell or tissue is cultured in the 3D matrix or 3D medium in the presence of a stress pathway inducing compound or method or in the presence of a culture medium comprising anisomycin. Stress pathway inducing compounds include but are not limited to anisomycin or any other p38 inducing compounds. For example anisomycin can be added to the culture medium before or during culturing the cell or tissue. The amount of anisomycin in the culture medium may vary depending on the cells and culture medium, including but not limited to 15 ng/mL - 250 ng/mL, 50 ng/ml - 500 ng/mL, 100 ng - 1 ug/mL, 1 ug/mL - 10 ug/mL, 5 - 50 µg/mL, 10 - 40 µg/mL, or 15 - 35 µg/mL., e.g. 25 µg/mL. In one embodiment p38 expression can be induced by culturing the cell or tissue in the 3D matrix or 3D medium in an air-liquid interface culture. In one embodiment liquid culture medium is added on or to the matrix comprising embedded cells or tissue, e.g. in a way that the media surrounds the matrix including the top of the matrix. In one embodiment the matrix is in an air-liquid interface culture or the ex vivo culture is performed as an air-liquid interface culture. “An air-liquid interface cell culture” refers to a culture, wherein a lower part of the cells and/or matrix comprising cells is grown in contact with a culture medium, and an upper part of the cells and/or matrix comprising cells is exposed to the air. In one embodiment the matrix can be lifted and the liquid medium can be changed if need arises. Any mammalian cells are suitable for an air-liquid interface cell culture.

In one embodiment of the method of maintaining the presence or activity of ERα the cell or tissue is cultured in the 3D matrix or 3D medium in the presence of an epigenetic pathway modulating compound that optionally influences H3K27 methylation. Suitable epigenetic pathway modulating compounds that optionally influence H3K27 methylation include but are not limited to an EZH2 inhibitor (e.g. GSK-126). In one embodiment of the invention the cell or tissue is cultured in the 3D matrix or 3D medium in the presence of an inhibitor of the methyltransferase enhancer of zeste homolog 2 (EZH2) or in the presence of a culture medium comprising an inhibitor of the methyltransferase EZH2. EZH2, a histone methyltransferase and a catalytic component of PRC2, catalyzes tri-methylation of histone H3 at Lys 27 (H3K27me3) to regulate gene expression through epigenetic machinery. Numerous studies have highlighted the role of EZH2 in cancer development and progression. Through modulating critical gene expression, EZH2 promotes cell survival, proliferation, epithelial to mesenchymal, invasion, and drug resistance of cancer cells. The tumor suppressive effects of EZH2 have also been identified. In the present invention, an inhibitor of EZH2 can be utilized for maintaining the presence or activity of ERα.

The present invention also concerns a method of maintaining a luminal epithelial phenotype and/or luminal epithelial cell identity of a mammalian cell in an ex vivo cell or tissue culture. In one embodiment of the present invention the matrix is capable of preserving a luminal epithelial phenotype and/or luminal epithelial cell identity of a cell or tissue embedded in said matrix.

As used herein “maintaining” or “preserving” does not necessarily mean only maintaining or preserving 100% of a luminal epithelial phenotype and/or cell identity of an ex vivo cell, cells or tissue compared to the luminal epithelial phenotype and/or cell identity of an in vivo cell, cells or tissue, but also maintaining or preserving at least some, e.g. at least 10%, 20%, 30%, 40%, 50%, 60%, 70%, 80%, 90% or 95% of the luminal epithelial phenotype and/or cell identity of the cells or tissue, or at least 10%, 20%, 30%, 40%, 50%, 60%, 70%, 80%, 90% or 95% of the cells having the luminal epithelial phenotype and/or cell identity in a tissue or culture. Depending on the studies carried on ex vivo cell or tissue cultures in 3D matrices or 3D media said cultures can be needed or last for example few hours to months. In one embodiment a luminal epithelial phenotype and/or cell identity is maintained for at least two, three, four, five, six, seven, eight, nine or ten days (e.g. at least one, two, thee or four weeks) in an ex vivo culture.

A luminal epithelial phenotype of a cell and/or luminal epithelial cell identity may be characterized by any suitable method known to a person skilled in the art including but not limited to a gene expression profile, staining, immunostaining and/or fluorescent tagging. In one embodiment of the invention the method comprises determining the luminal epithelial phenotype and/or luminal epithelial cell identity of the cell embedded in the matrix e.g. by a gene expression profile, staining, immunostaining and/or fluorescent tagging. Gene expression profiling, staining, immunostaining and/or fluorescent tagging are common methods known to a person skilled in the art and described in laboratory manuals of the field. Gene expression profiling is the measurement of the activity (i.e. the expression) of one or more genes, even hundreds or several thousands of genes e.g. at once. Biomolecular methods suitable for gene expression profiling include but are not limited to northern blot, RNA in situ hybridization, fluorescent in situ hybridization, reverse transcription PCR (RT-PCR), qRT-PCR, DNA microarray, tiling array, and RNA-sequencing. Furthermore, a western blotting method may be used by a skilled person to determine the amount of expressed proteins. Stains and dyes can be used to define e.g. tissues, cell populations, cells, or organelles within cells. E.g. DNA, RNA, protein, lipid, or carbohydrate specific dyes can be used for studying specific compounds. Immunostaining can be used for detecting a specific protein, e.g., in a tissue, cell population, cell, or in a sample.

As used herein “phenotype of a cell” refers to the conglomerate of multiple cellular processes involving gene and protein expression that result in the elaboration of a cell’s particular morphology and function. As used herein “a cell identity” refers to e.g. gene expression dynamics, size, growth rate, and any functional identity of a cell, as well as rates of transcription, translation or degradation in said cell to mentioned few examples. The scopes expressions “phenotype of a cell” and “a cell identity” can overlap.

It is very difficult to preserve a luminal epithelial phenotype or cell identity of cells in cultures, especially tumor cells or tissues, including but not limited to mammary, prostate, ovary, kidney, liver, bladder, pancreas or cartilage cells or tissues. As an example, about 80% of breast cancers are luminal type. Moreover, luminal cells are considered as the cells of the origin for the vast majority of breast cancers. Furthermore, both basal and luminal cells can serve as cells of origin for prostate cancer. Primary prostate cancer nearly always has a luminal phenotype characterized by atypical glands, strong androgen receptor (AR) signaling, and an absence of basal cells. About 60% of all ovarian tumors display high expression of estrogen receptors (ER) and display mutations resembling luminal breast cancers.

Suitable markers for determining e.g. the basal epithelial phenotype or identity of a cell include ΔNp63, cytokeratin 14 (CK14) and cytokeratin 5 (CK5). As an example, decreased basal phenotype or even lack of basal phenotype of a cell or tissue can be determined e.g. by a decreased level of ΔNp63, CK14 and/or CK5 or lack of one or more of said markers.

One or more suitable markers for determining e.g. the luminal epithelial cell identity or phenotype may be selected from the group consisting of notch, cytokeratin 8 (CK8) and cytokeratin 18 (CK18). As an example, the luminal epithelial phenotype can be determined e.g. by the presence of notch, CK8 and/or CK18 in a cell or tissue.

In one embodiment the luminal epithelial phenotype or luminal epithelial cell identity of the cell is characterized or determined by the presence of at least one or more cytokeratins optionally selected from the group consisting of notch, cytokeratin 8 (CK8) and cytokeratin 18 (CK18); the lack of one or more basal cytokeratins optionally selected from the group consisting of ΔNp63, cytokeratin 5 (CK5) and cytokeratin 14 (CK14); a gene expression profile associated with a luminal phenotype, and/or the lack of a gene expression profile associated with a basal phenotype.

The method of the present invention of maintaining the presence or activity of ER, a luminal epithelial phenotype and/or cell identity comprises culturing a cell or tissue in a 3D matrix with storage modulus of at least about 10 kPa, or in a 3D medium comprising said matrix. Also the present invention relates to a 3D matrix or 3D medium comprising the matrix for ex vivo culture, wherein said 3D matrix or 3D medium comprises one or more mammalian cells or tissues embedded in said 3D matrix or 3D medium, wherein the storage modulus of the matrix is at least about 10 kPa, optionally measured by dynamic rheology.

The present invention further concerns maintaining a luminal epithelial phenotype and/or cell identity, wherein the method comprises culturing a mammalian cell or tissue in a 3D matrix or 3D medium comprising said matrix, wherein the matrix is substantially gelled, bioinert and/or the mechanical stiffness of the matrix is at least about 0.1, 0.5, 1, 2, 3, 4, 5, 6, 7, 8, 9 or 10 Pa, optionally measured by dynamic rheology and presented as either storage modulus or elastic modulus; or at least about 0.1, 0.5, 1, 2, 3, 4, 5, 6, 7, 8, 9 or 10 kPa, optionally measured by dynamic rheology and presented as either storage modulus or elastic modulus. Also, the present invention relates to a 3D matrix or 3D medium comprising the matrix for ex vivo culture, wherein said 3D matrix or 3D medium comprises one or more mammalian cells or tissues embedded in said 3D matrix or 3D medium, wherein the matrix is substantially gelled, bioinert and/or the mechanical stiffness of the matrix is at least about 0.1, 0.5, 1, 2, 3, 4, 5, 6, 7, 8, 9 or 10 Pa, optionally measured by dynamic rheology and presented as either storage modulus or elastic modulus; or at least about 0.1, 0.5, 1, 2, 3, 4, 5, 6, 7, 8, 9 or 10 kPa, optionally measured by dynamic rheology and presented as either storage modulus or elastic modulus.

As used herein, “a matrix” refers to a material to support the maintenance, growth and/or culturing of cells or tissues. In one embodiment the cells or tissues are embedded in the matrix for ex vivo culture. The physical and/or chemical nature of the matrix can play a decisive role in the present invention. The surprising finding of the present invention is that the stiffness of the matrix has to be high enough to maintain the presence or activity of an estrogen receptor and/or the cell or tissue phenotype. On the other hand, when the matrix has a very high stiffness and/or high concentration of the gelled material has been utilized, problems to embed the cells and tissues to the matrix can arise. This limits the practical gel concentrations and the available stiffnesses allowed by different polymers.

Storage modulus of the matrix can be at least about 10 kPa. The matrix can have storage modulus of about 10 - 700 kPa, 10 - 600 kPa, 10 - 500 kPa, 10 - 400 kPa, 10 - 350 kPa, 10 - 300 kPa, 10 - 250 kPa, 10 - 200 kPa, 10 - 150 kPa, 10 - 140 kPa, 10 - 130 kPa, 10 - 120 kPa, 10 - 110 kPa, 10 - 100 kPa, 10 - 90 kPa, 10-80 kPa, 10 - 70 kPa, 10 - 60 kPa, 10 - 50 kPa, or 10 - 40 kPa e.g. for a mammal, human or mouse cell or tissue. On the other hand the matrix can have storage modulus of about 40 - 700 kPa, 40 - 600 kPa, 40 - 500 kPa, 40 - 400 kPa, 40 - 350 kPa, 40 - 300 kPa, 40 - 250 kPa, 40 - 200 kPa, 40 - 150 kPa, 40 - 140 kPa, 40 -130 kPa, 40 - 120 kPa, 40 - 110 kPa, 40 - 100 kPa, 90 - 700 kPa, 90 - 600 kPa, 90 - 500 kPa, 90 - 400 kPa, 90 - 300 kPa, 90 - 250 kPa, 90 - 200 kPa, 90 - 150 kPa, 90 - 140 kPa, 90 - 130 kPa, 90 - 120 kPa, 90 - 110 kPa or 90 - 100 kPa (such as 120 - 700 kPa, 120 - 600 kPa, 120 - 500 kPa, 120 - 400 kPa, 120 - 300 kPa or 120 - 200 kPa) e.g. for a mammal, human or mouse cell or tissue. In one embodiment of the invention storage modulus of the matrix is at least about 11 kPa, at least about 12 kPa, at least about 13 kPa, at least about 14 kPa, at least about 15 kPa, at least about 16 kPa, at least about 17 kPa, at least about 18 kPa, at least about 19 kPa, at least about 20 kPa, at least about 21 kPa, at least about 22 kPa, at least about 23 kPa, at least about 24 kPa, at least about 25 kPa, at least about 30 kPa or at least about 35 kPa (e.g. for a mammal, rodent or mouse cell). In one embodiment storage modulus of the matrix is at least about 40 kPa, at least about 45 kPa, at least about 50 kPa, at least about 55 kPa, at least about 60 kPa, at least about 65 kPa, at least about 70 kPa, at least about 75 kPa, at least about 80 kPa, at least about 85 kPa, at least about 90 kPa, at least about 95 kPa, at least about 100 kPa, at least about 110 kPa, at least about 120 kPa, at least about 130 kPa, at least about 140 kPa, at least about 150 kPa, at least about 200 kPa, at least about 250 kPa, at least about 300 kPa, at least about 350 kPa, at least about 400 kPa, at least about 450 kPa, or at least about 500 kPa (e.g. for a human, rodent or mouse cell).

In one embodiment the mechanical stiffness or storage modulus is determined or calculated from the matrix without the cells or tissue, or calculated for the matrix with the cells or tissue. “Mechanical stiffness” refers to the resistance of an elastic body to deflection or deformation by an applied force. In one embodiment, mechanical stiffness can be observed for example by using dynamic rheology, where the storage modulus G′ indicates the matrix stiffness and elasticity, whereas the loss modulus G″ relates to the viscous losses. Ideally in gels G′~ ω⁰ and G″~ ω⁰, where (ω is the frequency. In one embodiment of the invention mechanical stiffnesses are compared using elastic modulus, E, which can be calculated e.g. by utilizing the following equation: E=2(1-v)G, where G can be either the storage modulus, G′, or complex modulus

$$G * \left(G * = \sqrt{{{G}^{′}}^{2} + {{G}^{″}}^{2}}\right)$$

obtained from the dynamic rheology and v is the Poisson’s ratio, which is 0.5 for icompressible materials like ideal gels.

In one embodiment of the invention, when the storage modulus of the matrix is at least 10 kPa, the elastic modulus, E, of the matrix is at least about 20 kPa, 25 kPa, 30 kPa, 35 kPa, 40 kPa, 45 kPa, 50 kPa, 55 kPa, 60 kPa, 65 kPa, 70 kPa, 75 kPa, 80 kPa, 85 kPa, 90 kPa, 95 kPa, 100 kPa, 110 kPa, 120 kPa, 130 kPa, 140 kPa, 150 kPa, 160 kPa, 170 kPa, 180 kPa, 190 kPa, 200 kPa, 210 kPa, 220 kPa, 230 kPa, 240 kPa, 250 kPa, 260 kPa, 270 kPa, 280 kPa, 290 kPa, 300 kPa, 310 kPa, 320 kPa, 330 kPa, 340 kPa, 350 kPa, 360 kPa, 370 kPa, 380 kPa, 390 kPa, 400 kPa, 410 kPa, 420 kPa, 430 kPa, 440 kPa, 450 kPa, 460 kPa, 470 kPa, 480 kPa, 490 kPa, or at least about 500 kPa.

The mechanical stiffness or storage modulus of the matrix can be measured by any method suitable for measuring stiffnesses including but not limited to tensile testing, compression testing, atomic force microscopy, micropipette aspiration, acoustic methods and a storage modulus using dynamic rheology. In one embodiment the mechanical stiffness or storage modulus of the matrix is measured by dynamic rheology. Rheology is a method that provides quantitative parameters that define how a material will deform as a function of force, time, and spatial orientation. The critical quantities are measured in rheology to determine the viscoelastic properties of hydrogels are stress, strain, elastic modulus, and viscosity. Stress is the ratio of force (F) to the area (A) in which that force is exerted, expressed in N/m² or Pascal (Pa) units. When the applied stress is parallel to the surface, it is called shear stress. When the applied stress is perpendicular to the surface is called elongation or compressive stress, depending on the stress direction. The strain is a geometrical quantity used to quantify the fractional amount of deformation (the degree to which the material deforms) in a given material and is unitless. The ratio of stress to strain is defined as the elastic modulus of a solid in the elastic regime. The ratio of stress to the rate of strain (or flow rate), defines the viscosity for a liquid in the linear regime. Flow and deformation of materials under applied forces can be routinely measured, e.g., by a rheometer. In one embodiment of the invention dynamic oscillatory rheology measurements are performed to determine the mechanical properties of the matrix of the present invention. Oscillatory rheological measurements can comprise, e.g., one, two or three of the following parts: 1) time sweeps (e.g., performed at 1% strain) to follow the gelation or to confirm the stability of a gel, matrix or mixture; 2) frequency sweeps (e.g., at 1% strain or 0.3 Pa stress); 3) strain sweeps (e.g., at an angular frequency 1.0 rad/s) to confirm the linear viscoelastic region for oscillatory measurements. The gelation time and/or stabilities can be determined, e.g., based on the storage elastic modulus (G′) and/or loss modulus (G″) and can be presented by the unit Pascal (Pa). In one embodiment the stiffness (e.g., storage modulus G′) values for the matrices (e.g., substantially gelled materials) are determined after reaching the equilibrium under rheological measurement conditions.

In a very specific embodiment, rheology is carried out using TA Instruments AR2000 stress-controlled rheometer and a Peltier heated plate. In a further embodiment when utilizing TA Instruments AR2000 stress-controlled rheometer and a Peltier heated plate gap temperature compensation is set to 0.7 µm/°C and normal force control is used with matrices. The measurements can be performed, e.g, at two different temperatures such as 20° C. and 37° C.

In one embodiment the matrix is substantially gelled. Gels or substantially gelled materials refer to materials that have fully or partial levels of elastic behavior whereas fluids not belonging to the scope of gels or substantially gelled materials do not have elastic component. In more detail, whether the matrix is made of or comprises substantially gelled material can be observed for example using dynamic rheology, where the storage modulus G′ indicates the gel stiffness and elasticity, whereas the loss modulus G″ relates to the viscous losses. Ideally in gels G′~ ω⁰ and G″~ ω⁰, where ω is the frequency. By contrast in ideal viscous fluid state, in others words, in the complete absence of gelation, G′~ ω² and G″~ ω¹. Depending on the materials and conditions, the gelation can slowly develop and the gelation can also be incomplete, where the said ideal viscous fluid behavior is not observed. This is what is meant by substantially gelled material which have partially gelled signature in dynamic rheology. In one embodiment of the present invention the gelation or substantial gelation can be achieved by dispersing polymers in an aqueous medium and thereby forming chemical and/or physical networks. In a specific embodiment of the invention colloidal gels, such as using nanocellulose, can be utilized in the matrix.

In one embodiment of the invention the matrix comprises or is made of viscoelastic material to support the maintenance, growth and/or culturing of cells. More specifically, the group of viscoelastic materials includes but is not limited to a range of viscoelastic liquids and viscoelastic solids. As used here, “viscoelastic” refers to the property of materials that show both viscous and elastic characteristics when deformed. Viscosity is the measure of a material’s resistance to flow. Materials with low viscosity flow (e.g., water), whereas those with large viscosity show resistance to flow (e.g., honey). Elasticity refers to the property of materials to return to their original shape after deformation. Purely viscous material resists shear flow and ideally strains linearly with time when stress is applied and does not return to its original shape when stress is removed. Purely elastic material strains simultaneously when stressed and returns back to the original shape immediately when stress is removed. As used herein, “solid” refers to a state of the matter, which can support its weight and retain its shape or volume in a container when turned upside down, it does not flow. As used herein, “liquid” flows and takes on the shape of its container. Hydrogels are a type of viscoelastic materials, which can be used as the matrix or in the matrix of the present invention. As used herein, “hydrogel” is a three-dimensional (3D) polymer or colloidal network, which can retain large amounts of liquid, for example, water or cell culturing medium. A hydrogel can comprise one or more components. Depending on the flow behavior, the hydrogels can be classified as either viscoelastic liquids or viscoelastic solids.

In one embodiment of the invention the 3D medium and/or matrix is bioinert and/or substantially gelled. Indeed, in a specific embodiment the matrix does not comprise growth factors and/or the matrix does not comprise added growth factors. As used herein “added growth factors” refer to those growth factors, which can be added to a matrix of the present invention externally, i.e. they are not growth factors produced or released by the cells of a tissue comprised in the matrix of the present invention. As used herein “bioinert” or “biologically inert” refers to a 3D matrix, 3D medium or material which does not initiate a response or interact when allowed to contact with a cell or tissue.

In one embodiment of the invention the matrix comprises alginate, agarose, ovomucin, egg white, or any combination thereof. In one embodiment RGD-peptides are covalently linked to matrix (e.g., alginate) to equip the biopolymeric scaffold with adhesion sites. Biopolymers such as agarose (from red seaweeds), alginate (from brown seaweeds), and a commercial animal-free matrix GrowDex® lack cell adhesion sites and latent growth factors. Egg white is also of animal origin, but the heat-based polymerization of the matrix denatures the majority of its protein components, including the growth factors. Matrices made of alginate, agarose, ovomucin, egg white or any combination thereof can thus be considered as bioinert.

In a specific embodiment of the invention the matrices comprising alginate, agarose, ovomucin, or egg white are considered as luminal identity preserving matrices (LMx).

In one embodiment the matrix or a mixture for preparing the matrix comprises agarose at a concentration above about 20 mg/mL (mass of agarose powder per volume of the solvent). The matrix of the present invention or the mixture for preparing the matrix can comprise about 30 mg/mL or more agarose. In one embodiment of the invention the matrix or the mixture for preparing the matrix comprises about 35 mg/mL or more, about 40 mg/mL or more, about 45 mg/mL or more, about 50 mg/mL or more, about 55 mg/mL or more, about 60 mg/mL or more, about 65 mg/mL or more, or about 70 mg/mL or more agarose. In one embodiment of the invention low melting point agarose is utilized for the matrix or methods of the present invention. As known by a skilled person, there are several different agaroses such as low melting point agaroses commercially available. In a very specific embodiment, wherein low melting point agarose is used, the mixture solution for the matrix comprising agarose remains fluid at about 37° C. and will set rapidly at temperatures below 25° C.

In one embodiment the matrix or the mixture for preparing said matrix comprises alginate, ovomucin, and/or egg white optionally or for example at a concentration above about 20 mg/mL. The matrix of the present invention or the mixture for preparing said matrix can comprise about 30 mg/mL or more alginate, ovomucin, and/or egg white. In one embodiment of the invention the matrix comprises about 35 mg/mL or more, about 40 mg/mL or more, about 45 mg/mL or more, about 50 mg/mL or more, about 55 mg/mL or more, about 60 mg/mL or more, about 65 mg/mL or more, or about 70 mg/mL or more alginate, ovomucin, and/or egg white.

In some embodiments concerning the matrix which is substantially gelled, bioinert and/or the mechanical stiffness of the matrix is one or more selected from the group consisting of: at least about 0.1, 0.5, 1, 2, 3, 4, 5, 6, 7, 8, 9 or 10 Pa optionally measured by dynamic rheology and presented as either storage modulus or elastic modulus; at least about 0.1, 0.5, 1, 2, 3, 4, 5, 6, 7, 8, 9 or 10 kPa optionally measured by dynamic rheology and presented as either storage modulus or elastic modulus, or the method related thereto, the matrix or a mixture for preparing said matrix can comprises agarose, alginate, ovomucin, and/or egg white at a concentration e.g. less than about 20 mg/mL, about 0.1 mg/mL or more, about 0.5 mg/mL or more, about 1 mg/mL or more, about 2 mg/mL or more, about 3 mg/mL or more, about 4 mg/mL or more, about 5 mg/mL or more, about 6 mg/mL or more, about 7 mg/mL or more, about 8 mg/mL or more, about 9 mg/mL or more, about 10 mg/mL or more, about 15 mg/mL or more.

In an embodiment of the invention in addition to alginate, agarose, ovomucin, and/or egg white, the matrix or a mixture for preparing the matrix may comprise water, a physiological salt solution (e.g. PBS), and/or one or more culturing medias. In a very specific embodiment the matrix or mixture comprises alginate, agarose, ovomucin and/or egg white, and a physiological salt solution.

In one embodiment of the method of the present invention, the mechanical stiffness or storage modulus of the matrix under suitable culture conditions is achieved by using external mechanical compression or magnetic compression. Indeed, an external mechanical compression such as magnetic compression can be used for increasing the mechanical stiffness of the 3D matrix or 3D medium during ex vivo culture.

For example, mammalian cells (e.g. mouse or human cells) can be cultured under external mechanical or magnetic compression, and then the matrix can have under said culture conditions the mechanical stiffness or storage modulus of about 10 -700 kPa, 10 - 600 kPa, 10- 500 kPa, 10 - 400 kPa, 10 - 350 kPa, 10 - 300 kPa, 10 - 250 kPa, 10 - 200 kPa, 10 - 150 kPa, 10 - 140 kPa, 10 - 130 kPa, 10 - 120 kPa, 10 - 110 kPa, 10 - 100 kPa, 10- 90 kPa, 10 - 80 kPa, 10 - 70 kPa, 10-60 kPa, 10 - 50 kPa, or 10 - 40 kPa (e.g. for a mammal, human or mouse cell or tissue); or about 40 - 700 kPa, 40 - 600 kPa, 40 - 500 kPa, 40 - 400 kPa, 40 - 350 kPa, 40 - 300 kPa, 40 - 250 kPa, 40 - 200 kPa, 40 - 150 kPa, 40 - 140 kPa, 40 -130 kPa, 40 - 120 kPa, 40 - 110 kPa, 40 - 100 kPa, 90 - 700 kPa, 90 - 600 kPa, 90 - 500 kPa, 90 - 400 kPa, 90 - 300 kPa, 90 - 250 kPa, 90 - 200 kPa, 90 - 150 kPa, 90 - 140 kPa, 90 - 130 kPa, 90 - 120 kPa, 90 - 110 kPa or 90 - 100 kPa (such as 120 - 700 kPa, 120 - 600 kPa, 120 - 500 kPa, 120 - 400 kPa, 120 - 300 kPa or 120 - 200 kPa, or 100 - 700 kPa, 100 - 600 kPa, 100 - 500 kPa, 100 - 400 kPa, 100 - 300 kPa or 100 - 200 kPa) e.g. for a mammal, human or mouse cell or tissue. In one embodiment of the invention the cell or tissue can be cultured under external mechanical or magnetic compression, and then the matrix can have the mechanical stiffness or storage modulus of at least about 10 kPa, at least about 11 kPa, at least about 12 kPa, at least about 13 kPa, at least about 14 kPa, at least about 15 kPa, at least about 16 kPa, at least about 17 kPa, at least about 18 kPa, at least about 19 kPa, at least about 20 kPa, at least about 21 kPa, at least about 22 kPa, at least about 23 kPa, at least about 24 kPa, at least about 25 kPa, at least about 30 kPa, at least about 35 kPa, at least about 40 kPa, at least about 45 kPa, at least about 50 kPa, at least about 55 kPa, at least about 60 kPa, at least about 65 kPa, at least about 70 kPa, at least about 75 kPa, at least about 80 kPa, at least about 85 kPa, at least about 90 kPa, at least about 95 kPa, at least about 100 kPa, at least about 110 kPa, at least about 120 kPa, at least about 130 kPa, at least about 140 kPa, at least about 150 kPa, at least about 200 kPa, at least about 250 kPa, at least about 300 kPa, at least about 350 kPa, at least about 400 kPa, at least about 450 kPa, or at least about 500 kPa (e.g. for a human, rodent or mouse cell).

A cell such as a mammalian cell or a tissue such as a mammalian tissue is cultured in methods of the present invention and can be embedded in the matrix of the present invention. As used herein “a tissue” is an ensemble of two or more cells and their stromal component or extracellular matrix from the same origin. In other words, a tissue comprises at least two cells, which can be different types, and their stromal component or extracellular matrix from the same origin, e.g. the same organ. In one embodiment the mammalian tissue comprises tumor cells and/or non-tumor (i.e., normal) cells. Tumor cells have abnormal cell growth with the potential to invade or spread. In one embodiment of the invention the tissue is a primary tissue, e.g. a epithelial, connective, muscular or nervous tissue, or any combination thereof.

A mammalian cell or cells utilized in any method, 3D matrix or 3D medium of the present invention may be any cell or combination of any cells suitable for ex vivo cultures. Said cell(s) may be selected, e.g., from a non-tumor (i.e. normal) cell, tumor cell, cell from a cell line and a combination of a non-tumor and tumor cell or cell line.

The methods and tools of the present invention allow to explore therapeutic interventions, drug discovery, biomarker, and ERα signaling studies, genetics and/or biochemistry of all type of cells, in one specific embodiment luminal cells. In one embodiment of any method, 3D matrix or 3D medium of the invention, the cell is selected from the group consisting of a mammary cell, cell of the prostate, chondrocyte, kidney cell, pancreatic cell, bladder cell and ovarian cell, or the tissue comprises cells selected from the group consisting of mammary cells, cells of the prostate, chondrocytes, kidney cells, pancreatic cells, bladder cells and ovarian cells.

In one embodiment of the invention the mammalian cell or tissue is selected from the group consisting of a human, rodent, mouse, murine, hamster, rabbit, swine, dog, and cat cell or tissue.

As used herein “ex vivo” refers to that which takes place outside an organism. In one embodiment of the present invention the matrix for ex vivo culture is an in vitro matrix or the ex vivo culture of the present invention is an in vitro culture. As used herein “in vitro” refers to studies or cultures that are conducted using components of an organism (e.g. molecules, micro-organisms, cells, tissues, cell lines) that have been isolated from their usual biological surroundings. In contrast to “in vitro”, studies conducted in living beings are called in vivo studies.

In one embodiment of the invention the 3D matrix or 3D medium is for 3D tissue culture applications ex vivo. As used herein “3D tissue culture” refers to a three-dimensional tissue culture, i.e. an artificially created ex vivo or in vitro environment in which cells are permitted to grow or interact with their surroundings in all directions (i.e. three dimensions). 3D cultures aim to mimic the conditions and surrounding of cells in vivo. “3D medium” of the present invention comprises the matrix, one or more mammalian cells or tissues embedded in said matrix and optionally further agents, or “3D medium” consists of the matrix and one or more mammalian cells or tissues embedded in said matrix. In addition to the 3D matrix and 3D medium, the present invention further concerns a system for ex vivo culture, wherein the system comprises one or more mammalian cells or tissues embedded in a 3D matrix or 3D medium comprising said matrix. In one embodiment the system further comprises suitable tools for ex vivo culture selected from the group including but not limited to a culture plate or container, tools for obtaining en external or magnetic compression (e.g., one or more magnets and/or a metallic grid), tools for air-liquid interface culture (e.g. a cell culture insert and/or metallic grid), liquid medium for culturing, and any combination thereof.

In one embodiment of the invention, the 3D matrix, 3D medium or system is capable of maintaining or substantially maintaining the presence or activity of ER, a luminal epithelial phenotype and/or luminal epithelial cell identity of the cell or tissue embedded in said 3D matrix or 3D medium. In one embodiment the 3D matrix, 3D medium or system is for the method of the present invention for maintaining or substantially maintaining the presence or activity of ER, luminal epithelial phenotype and/or cell identity of a cell or tissue embedded in the 3D matrix or 3D medium.

Also, the present invention concerns use of the 3D matrix, 3D medium or system of the present invention for ex vivo culture of a mammalian cell, drug discovery methods, biomarker studies and/or estrogen receptor (ER) signaling studies.

The method of the present invention for preparing the 3D matrix or 3D medium of the present invention comprises embedding a cell or tissue (e.g. a mouse or human cell or tissue) in a matrix and optionally allowing the matrix to become substantially gelled.

As used herein “embedding” or “embedded” refers to a situation wherein at least one cell, some of the cells or at least part of the tissue is within the matrix. In a specific embodiment of the invention all cells or the tissue are within the matrix. An embedded tissue or embedded cells in a matrix may be obtained e.g. by placing the cell(s) or the tissue in the matrix. Alternatively, the liquid, substantially gelled or viscous matrix may be added on the cells or tissue, thereby forming the embedded cells or tissue. Indeed, in one embodiment when preparing the matrix, the matrix is first in a liquid, substantially gelled or viscous form enabling easy embedding of a cell or tissue. In a further embodiment the matrix may be solidified or substantially gelled e.g. at 10° C. - +37° C. such as at a room temperature (from 15 to below 25° C. (the European Pharmacopoeia).

In one embodiment of the invention the alginate, agarose, ovomucin, egg white or any combination thereof is in the form of a powder and optionally it is dispersed in water or a physiological salt solution (e.g. PBS) e.g. at room temperature. In one embodiment after dispersing the powder in water or a physiological salt solution, the obtained matrix mixture is heated until the powder is essentially dissolved. In one embodiment the egg white used for the matrix is in a form obtained from an egg, optionally filtered through a sinter.

A method of the present invention for culturing a cell or tissue (e.g., a mouse or human cell or tissue) ex vivo comprises culturing a cell or tissue (e.g., a mouse or human cell or tissue) in the matrix of the present invention in suitable conditions. Suitable conditions, such as temperature, selection of nutrients, gases, humidity and the like, as well as different incubators are within the knowledge of a skilled person and can be selected to provide an economical and effective method with the tissue or cells in question. Temperatures during culturing may range within the knowledge of a skilled person, although the optimal temperature may depend on the tissue or cells. In a specific embodiment the culturing temperature is from about 35° C. to about 39° C., e.g. around 37° C. Culture medium may be used in the method or ex vivo culture of the present invention and it can be any suitable culture medium known to a person skilled in the art, e.g. selected from the group consisting of DMEM, RPMI, EMEM, L-15, DMEM/F12, MCDB170, Ham’s F12, and MammoCult.

Results of the present disclosure reveal that the 3D culture with a matrix having higher stiffness significantly supported the nuclear localization of ERα. The findings of the present disclosure further reveal that the nature of the physical and chemical microenvironment, together with the nature of genetic mutations, is the main causative determinant of the luminal tumor phenotype. The loss of appropriate physical and chemical microenvironment may explain the often-observed failure in tumor cell growth and alterations in their phenotype in ex vivo 3D models of the prior art.

The very specific embodiment of the present disclosure shows that microenviron-mental mechanical stress of breast cancer cells triggers luminal cell differentiation towards estrogen receptor expression. The luminal cell maturation is mediated through stress activated protein kinase.

The results of the present disclosure also show that expression of estrogen receptor is different from its activation. This is supported by the cytoplasmic localization of ERα in tumor fragments, which were cultured in soft matrix. This was supported by the gene expression profiling, which revealed that despite the receptor expression, its mediated functions were absent in the soft matrix. For example MMEC fragments cultured in stiff matrix (e.g., agarose, e.g., 70 mg/ml agarose) shared a similar expression profiles with human ERα positive tumors.

Indeed, the present invention reveals that with the right matrix and/or stiffness thereof the creation of mammal explant models preserving the ERα,a luminal epithelial phenotype and/or cell identity is enabled.

It will be obvious to a person skilled in the art that, as the technology advances, that the inventive concept can be implemented in various ways. The invention and its embodiments are not limited to the examples described below but may vary within the scope of the claims.

### EXAMPLES

### Materials and Methods

Agarose (UltraPure™ Low Melting Point Agarose, Invitrogen™, lot: 0000520356) solutions were prepared by first dispersing the agarose powder in 1×PBS at room temperature followed by heating the mixtures until the agarose was completely dissolved followed by cooling to desired temperature.

Collagen (Collagen I Rat tail, Corning) solutions were prepared by partly following Alternate Gelation Procedure for BD™ Collagen I, Rat tail. All the components and equipment were pre-cooled and kept on ice during the preparation. First 10 × PBS and water were mixed together. Then Collagen I rat tail stock solution was added and solution was mixed well again. Last 1M NaOH was added and mixed throughout, with pipette. Solution was used immediately or stored on ice 2-3 h prior the gelling.

Matrigel (Matrigel Basement Matrix Growth Factor Reduced, 8.8 mg/mL, Corning, lot:4174004) was thawed until it was liquid. All the components and equipment were pre-cooled and kept on ice during the preparation. Matrigel 8.8 mg/mL was used as it is or mixed with pre-cooled DMEM to obtain desired concentration.

Egg whites, from chicken egg, were removed from yolks and filter through a sinter to keep only the clear part. The egg white was divided in microcentrifuge tubes (a 1.5 mL) and stored in freezer (-20° C.). Samples were thawed prior the measurements.

GrowDex (15 mg/mL, UPM-Kymmene OYj) were used as they were.

Alginate and Alginate-RGD: Alginate (Alginic acid sodium salt, Aldrich, lot: MKBV5260V) and Alginate-RGD (Sodium Alginate MVG GRGDSP, NovaMatrix, NOVATACH, lot: BP-1730-06, BP-1802-04) powders were weighed in glass vials and 1XPBS was added. Solution were stirred and kept in the fridge over the night.

Ovomucin was extracted from chicken egg whites and prepared according to an established protocol (Omana and Wu, 2009, J Agric Food Chem 57, 3596-3603). Briefly, ovomucin was separated using isoelectric precipitation in the presence of 100 mM NaCl solution (pH adjusted to 6.0, kept at 4° C. overnight followed by centrifugation). Precipitated ovomucin was re-suspended in 500 mM NaCl, pH was adjusted to 6.0 again and kept at 4° C. overnight Dispersion was centrifuged and precipitated ovomucin was purified by dialysing against water overnight. Dialysed sample were freeze-dried under the vacuum. Solid ovomucin powder were dissolved in MilliQ water using Ultra-Turrax (IKA) mixer.

### Isolation of Biological Material and Culturing in 3D

Fresh tissue was obtained from elective breast cancer surgeries. Tissue was collected from both the tumor tissue and the area surrounding the tumor (non-tumor samples). Also tissue from reduction mammoplasties (RMP) were collected. From every tumor, one portion was taken for immunohistochemical analysis (IHC), for DNA/RNA/protein isolation, and the rest was used for isolation of fragments and 3D culture. For isolation of human breast cancer fragments, breast cancer tissue was incubated with 0.2% collagenase in MammoCult media produced by StemCell technologies (supplemented with 4 ug/mL heparin, MammoCult proliferation supplements, 0.1 µg/mL 10.000 UI/mL PenStrep, Amphotericin, and 25 µg/mL Gentamycin) with gentle shaking at 37° C. overnight. The resulting cell suspension was then centrifuged at 1,300 rpm for 3 min, washed once with MammoCult and plated in 8-chamber plates. Matrices used for the cultures were: Agarose (UltraPure™ Low Melting Point Agarose, Invitrogen™), Collagen (Collagen I Rat tail, Corning), Matrigel (Matrigel Basement Matrix Growth Factor Reduced, 8.8 mg/mL, Corning), Egg white, Ovomucine, GrowDex (15 mg/mL, UPM-Kymmene OYj), Alginate (Alginic acid sodium salt, Aldrich) and Alginate-RGD (Sodium Alginate MVG GRGDSP, NovaMatrix, NOVATACH). Ovomucin was isolated from chicken egg whites according to an established protocol (Omana and Wu, 2009, J Agric Food Chem 57, 3596-3603). Briefly, ovomucin was isolated from egg white using isoelectric precipitation in the presence of 100 mM NaCl solution at pH adjusted to 6.0. The resulting mixture was stored overnight at 4° C. in a refridgerator followed by centrifugation. The precipitate was re-suspended in 500 mM NaCl solution at pH 6.0 again and stored overnight at 4° C. The dispersion was centrifuged, and the precipitate was purified by dialyzing against water overnight. Dialysed sample was freeze-dried under the vacuum. For gelation, the solid ovomucin powder was dissolved in MilliQ (18 W) water using Ultra-Turrax (IKA) mixer.

Mouse primary mammary epithelial fragments were isolated with 0.2% collagenase for 1 h at +37° C. Media used for the mouse tissue was DMEM/F12 (Thermo Fisher Scientific) supplemented with 5% horse serum, 5 ug/mL Insulin, 1 µg/mL hydrocortisone, 10 ng/mL EGF, and 100 U PenStrep.

### Rheological Measurements

Rheology was carried out using TA Instruments AR2000 stress-controlled rheometer and a Peltier heated plate. 20 mm parallel steel plates were used, if a matrix sample without cells tended to show slippage 20 mm cross hatched parallel steel plates were used to prevent it. Matrix samples were covered with a sealing lid and the solvent trap as well as edges of the sealing lid were filled with silicon oil in order to prevent evaporation during the measurements. Gap temperature compensation was set to 0.7 µm/°C. Normal force control was used with agarose gels. The measurements were performed at two different temperatures viz. 20° C. and 37° C.

### Representative Example: Rheology

Dynamic oscillatory rheology measurements were performed to determine the mechanical properties of the gels (matrixes). A known amount of solid agarose powder was dissolved in 1×PBS according to the gelation procedure described. The pre-made agarose gel was placed on pre-heated 65° C. plate followed by 1.0 h gelling at 20° C., after which the temperature was kept at 20° C. or increased to 37° C. depending on the measurement. Collagen and Matrigel solutions were placed on pre-cooled 4° C. plate followed by 1 h gelling at 37° C. Egg white was placed on pre-cooled 4° C. plate followed by 1 h gelling at 57° C., 60° C. or 62° C., after which the temperature was decreased to 37° C. All the other samples were placed on plate at room temperature (20° C.) followed by the time sweep at 37° C. Usually measurement series consisted three parts: 1) Time sweeps were performed at 1% strain to follow the gelation or confirm the stability of the gels (except alginate 10% strain and alginate-RGD 1%, 5% or 10% strain). 2) Frequency sweeps were carried out at 1% strain or 0.3 Pa stress (except alginate 10% strain or 0.15 Pa or 0.3 Pa stress, and alginate-RGD 1%, 5% or 10% strain or 0.3 Pa or 0.5 Pa stress). 3) The linear viscoelastic region for oscillatory measurements were confirmed with strain sweeps at an angular frequency 1.0 rad/s.

The gelation time and stabilities were determined based on the storage elastic modulus (G′) and loss modulus (G″) and are presented in the unit Pascal (Pa). The samples before gelation show higher loss modulus (G″) values compared to that of storage modulus. Upon gelation a cross-over between G′ and G″ occurred and in their gel state G′ display an order of magnitude higher values than that of G″ . The complete gelation and equilibration displayed constant G′ and G″ values. The stiffness (i.e. storage modulus G′) values for the gels were determined for all the gels after reaching equilibrium under rheological measurement conditions. Based on the rheological properties the gels were classified into strain-softening and strain-stiffening.

### Sample Preparation for Rheological Measurements

Agarose in situ: Agarose powder was weighed in glass vial and 1×PBS was added. Solution was heated until agarose was dissolved. Solution was pipeted immediately at 65° C. on to the rheometer plates. Temperature was decreased to 20° C. and measurement (gelation, time sweep) was started. After gelation, the temperature was increased to 37° C. and frequency and strain sweep were measured after 1h time sweep at 37° C. (stabilization). Some concentrations were studied only at 20° C. without the 37° C. time sweep and frequency and strain sweeps are done at 20° C.

Agarose molded: Agarose powder was weighed in glass vial and 1×PBS was added. Solution was heated until agarose was dissolved. Solution was pipeted on the steel mold and let cool down 1h at 20° C. to form a gel. Gel was measured at 37° C.

Alginate and alginate-RGD: Alginate powders were weighed in glass vial and1XPBS was added. Solution was stirred and kept in the fridge overnight. Gel was pipeted on the rheometer and measurements were done at 37° C.

### Magnetic Force-Mediated Compression

Two magnets (Magnet Expert Ltd; #F643-SC) were used together with metallic grid on the top of the cell culture to create a vertical compression. The explant cultures had a round shape with a radius of Rₘₐₜᵣᵢₓ = 2.5 mm and an initial thickness of I₀ = 2.0 mm. The magnets together with the metallic grid applied a vertical compressive force of F_{compression} = 0.724 N (0.711 N; calculated based on Abbott et al., (2007, IEEE T-RO 23, 1247-1252) and 0.013 N; the top magnet/grid weight). Compression pressure (σ_{compression}) by the magnets is defined by: σ_{compression} = F_{compression}/A, where A is the cross-sectional area of each explant culture. The cultures were compressed by σ_{compression} = 37 kPa. Based on the initial volume of each explant culture (V₀ = l₀πRₘₐₜᵣᵢₓ²), vertical strain

$$( {ε}_{\text{compression}} \approx \frac{Δ V}{{V}_{0}} = \frac{{σ}_{\text{compression}}}{K} ,$$

where ΔV ≈ ΔlπRₘₐₜᵣᵢₓ², Δl is the vertical compression-related thickness change), bulk modulus

$$( K = \frac{2 \left(1 + υ\right) G *}{3 \left(1 - 2 υ\right)} ,$$

K where u is the Poisson’s ratio of agarose: u = 0.44 is based on (Brewin et al. 2015, Ann Biomed Eng 43, 2587-2596) and |G∗| is initial complex modulus also called the initial absolute shear modulus obtained from dynamic rheology) the effective elastic modulus of the compressed matrix

$$( {E}_{\text{effective}} = \frac{{σ}_{\text{compression}}}{{ε}_{\text{compression}}} )$$

and effective shear modulus

$$( G {*}_{e f f e c t i v e} = \frac{{E}_{e f f e c t i v e}}{2 \left(1 + υ\right)} )$$

are calculated. The compressing of the LMx-Ag, with the concentration of 70 mg/mL, resulted in G∗_{effective} = 129 kPa and E_{effective}=373 kPa.

### Immunofluorescent Staining

For immunofluorescent analysis 3D cultured breast cancer fragments were fixed with 4% paraformaldehyde for 20 min at room temperature and washed three times with PBS. The tissue fragments were permeabilized with 0.25% Triton X-100 in PBS for 10 min at RT and blocked in IF buffer (7.7 mM NaN₃, 0.1% BSA, 0.2% Triton X-100, and 0.05% Tween 20 in PBS) supplemented with 10% (vol/vol) normal goat serum for1-1.5 h. The fragments were then incubated with the primary antibody diluted in blocking solution overnight at +4° C. Following incubation, fragments were washed three times with IF buffer (20 min each wash) and then incubated with the appropriate Alexa Fluor secondary antibody diluted in IF buffer with 10% goat serum. After 60 min of incubation at RT, the fragments were washed with IF buffer as before and the nuclei were counterstained with Hoechst 33258 (Sigma). Slides containing tissue fragments were mounted with ImmuMount reagent. Images of the structures were acquired using a Leica TCS SP8 CARS confocal microscope using a HC PL APO CS2 40x objective.

TABLE 1

Antibodies

| | |
|---|---|
| Protein | IF |
| Keratin 8 | MMS-162P BioLegend |
| Keratin 14 | PRB-155P BioLegend |
| anti-p63 (ΔN) | #619002 BioLegend |
| Estrogen receptor α | (MC-20) sc-542 Santa Cruz, Millipore (06-935), ab16660 (IHC-IF) |
| Ki67 | NCL-Ki67 Leica Biosystems |
| p-p38 | #4511 Cell signaling |
| Phalloidin | Alexa-fluor 546 Thermo Fisher |

### DNA/RNA Sequencing and Data Analysis

Total RNA was isolated using RNeasy (Qiagen) or Trizol (Ambion) and DNAase removal step was performed after the isolation (Zymo research). 100 ng of total RNA was processed with ScriptSeq Complete Gold Kit is used for RNA-sequencing library preparation for next generation sequencing or with NEBNext Ultra Directional RNA Library Prep Kit for Illumina depending on the RNA integrity.

Ribosomal RNA was removed from the total RNA using Ribo-Zero™ Gold rRNA Removal Kit after which the RNA is fragmented chemically. During the cDNA synthesis the RNA is reverse transcribed using random hexamers with unique tagging sequences. Then, a terminal tagging oligonucleotide (TTO) is annealed to the 3′ end of the cDNA. The extension with DNA polymerase adds a second unique tagging sequence complementary to the TTO. The di-tagged cDNA is purified after which each sample is given a unique index barcode and amplified by PCR. The amplified library is purified with AMPure XP Beads. Finally, the library is assessed by using Agilent Bioanalyzer.

NEBNext Ultra Directional RNA Library Prep Kit for Illumina is used to generate cDNA libraries for next generation sequencing. First, the ribosomal RNA depleted sample (10 ng) is fragmented to generate inserts around 200 bp, and then primed with random primers. The first strand cDNA synthesis utilizes Actinomycin D, which inhibits the DNA polymerase activity of the reverse transcriptase increasing strand specificity. In the second strand cDNA synthesis dUTP labelled oligo nucleotides are incorporated to mark the second strand with uracils (U). The cDNA synthesis product is purified with Agencourt AMPure XP beads. Next, the cDNA is end-repaired, and adapter ligated utilizing dA-tailing. The adaptor ligated DNA goes through a bead-based size selection after which the final PCR enrichment takes place. At this point, each sample is given a unique index to enable pooling of multiple samples (multiplexing) for sequencing. During the high-fidelity PCR, USER (Uracil-specific Excision Reagent) enzyme cuts away the uracil strand preserving only the first strand. In addition, the loop adaptor is cut open to enable the PCR. The amplified library is then purified using AMPure XP Beads. Library quality is assessed by Bioanalyzer (Agilent DNA High Sensitivity chip) and library quantity by Qubit (Invitrogen). The next generation sequencing was performed with NextSeq 500 - Illumina instrument 75 PE giving 33 M reads/ sample. Gene set enrichment analysis 3.0 (Broad Institute) was used to analyze differences in gene expression profiles.

DNA was extracted from original tumor tissues and 3D cultured samples. The DNA integrity was measured using gel electrophoresis. TruSeq Amplicon Cancer Panel (TSACP, Illumina), which covers hotspot regions of 48 genes, was selected for the mutational profiling. Sequencing libraries were performed according to the manufacturer’s instructions and the samples were sequenced with MiSeq sequencer (Illumina). MiSeq Reporter was applied for the data--analysis and GATK tool was used for variant calling.

### Ethical Considerations

The described animal experiments have been processed through ethical review and they are covered under animal committee at the State Provincial Office of Southern Finland. The license numbers are ESAVI/15159/2019 and KEK19-002. The experiments, which involved human breast cancer tissue have ethical approval under the license number 243/13/03/02/2013/ TMK02 157. The study was approved by Helsinki University Hospital Ethical Committee. All patients, who participated to this study gave a written consent.

### Results

### 3D Patient Derived Explant Culture Platform (PDEC)

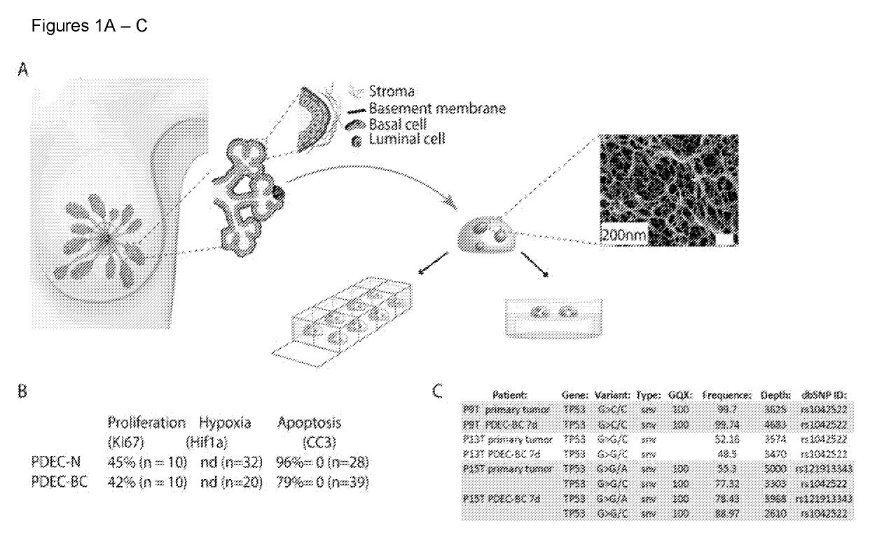

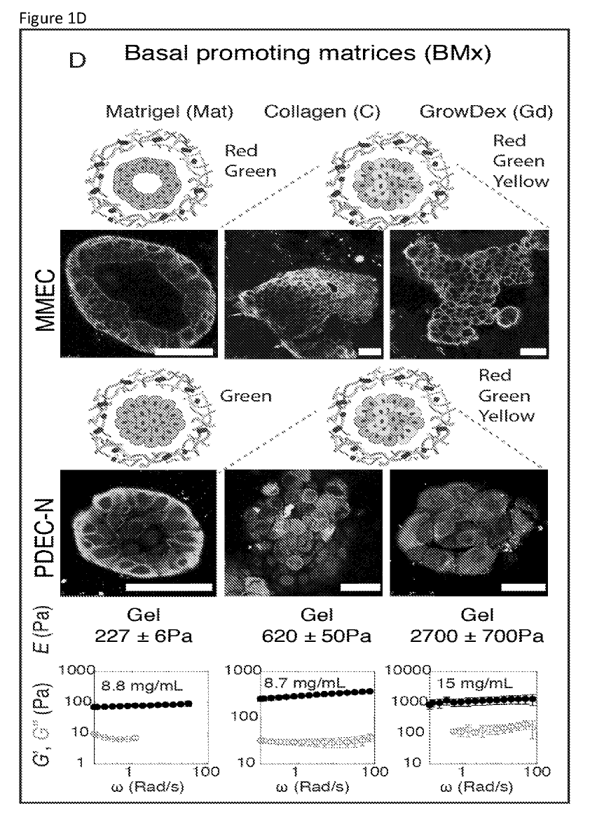

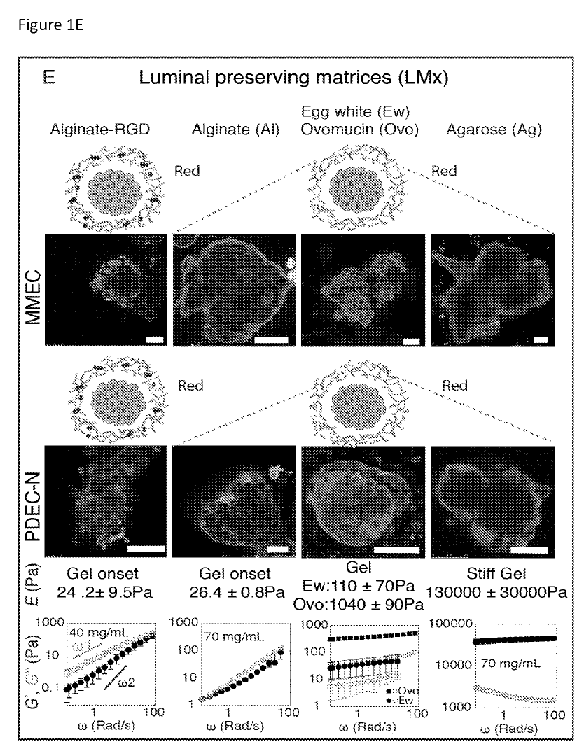

We have processed over 800 live breast cancer samples from elective surgeries and healthy breast tissue from reduction mammoplasty surgeries. These samples have been processed to living patient derived explant cultures (PDEC) (50-200 µm diameter sized epithelial fragments) grown in different 3D matrices (FIG. 1A). The PDECs grew in optimized medium for weeks and even months without losing viability. The explant viability was characterized by using markers for proliferation, apoptosis, and hypoxia after one week of culture (FIG. 1B). Also, some genetic features of the original tumors were retained in the explants, which was confirmed with DNA sequencing. The variants from the original tumors were observed to match with the variants in the cultured fragments (FIG. 1C). In the culturing studies, a specific aim was to monitor any changes in fragment cellular identity during the culture period revealing, if the model would be truly representative of the cell types present in the original sample. Eight different matrices were selected whether they are able to maintain the luminal phenotype of the fragments as desired or whether an unwanted change to the basal phenotype results. Normal tissues from the mouse mammary epithelial cells (MMEC) and PDECs from the human reduction mammoplasties (PDEC-N) were first used to test the normal responses of mammary tissue on diverse matrices. The cellular heterogeneity of fragments was monitored with immunofluorescent staining using specific cytokeratin expression markers for luminal (CK8) and basal cells (CK14).

### Comparative Example: Basal Cell Identity Promoting Matrices (BMx)

First, commonly used matrices were selected to explore whether they could allow maintaining the luminal cell identities of MMEC and PDEC-N upon culture. For that, results based on Matrigel (Mat), Collagen (C), and GrowDex (Gd) are shown in FIG. 1D. They were all applied using the concentrations allowed by the commercial providers (i.e., 8.8 \. mg/mL, 8.7 mg/mL, and 15 mg/mL, respectively). At these concentrations they all show clear gels where the rheological storage modulus is essentially independent of the frequency. Their stiffnesses (E) range from ca. 200 Pa to 3000 Pa. In all cases clear change from the luminal cell identities to basal identities were observed during one week culturing indicated by the CK8 and CK14 markers. Therefore, one can conclude that the MMEC and PDEC-N luminal cell identity cannot be maintained in the said common culture media, thus requiring new approaches.

### Luminal Cell Identity Preserving Matrices (LMx)

By contrast, luminal cell identity was maintained and observed in egg white (LMx-Ew), ovomucin (Ovo), in alginate (LMx-Al) or in agarose (LMx-Ag) hydrogels based on luminal cytokeratin expression (FIG. 1E). All these matrices, which preserved the luminal phenotype, were bioinert from their chemical properties. In order to reveal, whether the cell adhesion was responsible for the phenotype switch, we also used bioinert alginate, which was biofunctionalized with RGD sites. The phenotype remained, however, luminal despite the covalently attached adhesion sites. In rheological measurements, Ew, Ovo, and Ag formed well defined gels, where Ag additionally allowed reaching particularly high modulus E using a high concentration 70 mg/mL. Al and Alginate-RGD showed onset of gelation with the strorage modulus scales approximately with the first power of the frequency, i.e., it is not any more in the purely viscous state (FIG. 1E). Unlike in many other contexts, where the strain-stiffening has been shown to participate to the cell identity regulation, here the strain-stiffening did not seem to affect the luminal to basal phenotype conversion.

### Comparison of Cell Identity Preservation Upon Culturing Normal PDEC-N and Breast Cancer Tissues PDEC-BC

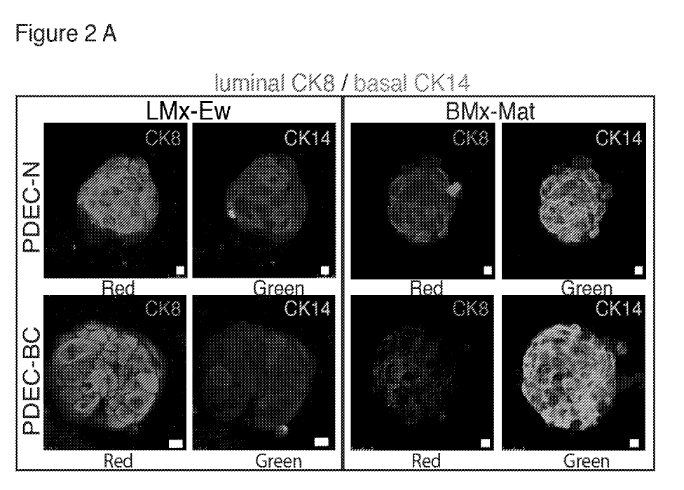

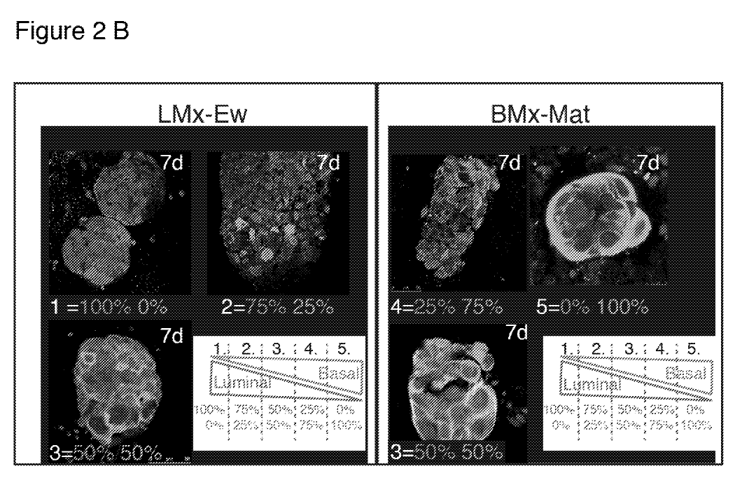

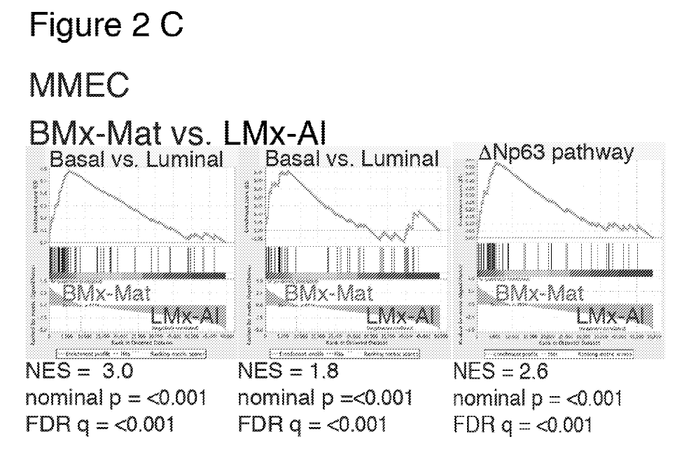

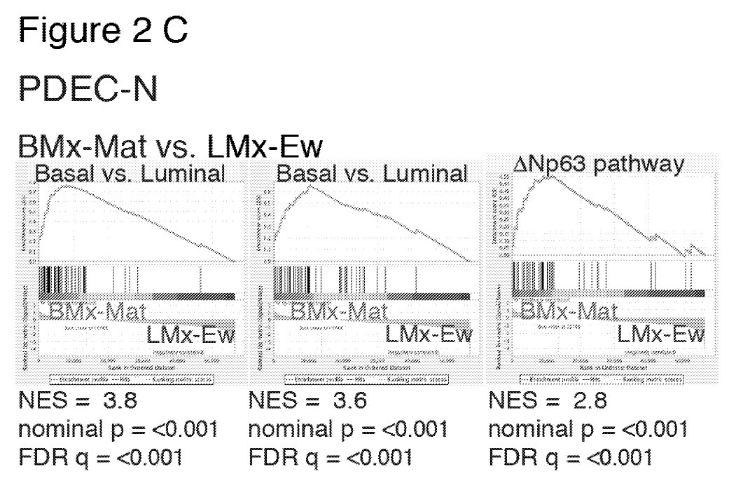

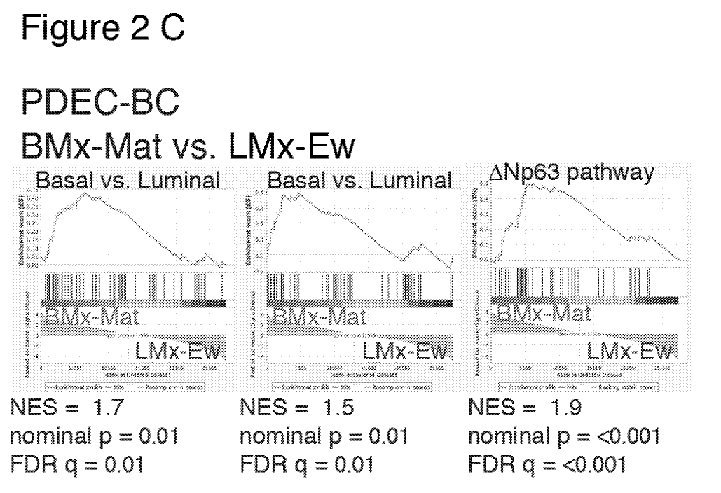

The matrix effect on the luminal phenotype preservation was also tested with PDEC-BC samples and compared to the results obtained using PDEC-N using two matrices, i.e., Ew and Mat (FIGS. 2A-B). In analogy with FIG. 1, Ew maintains the luminal cell identity for both tissues, whereas Mat promotes the basal cell identities.

### Transcriptional Profiling Confirmed the Effects From Different Culture Gels

To get an insight at the molecular level differences between the explants, which were grown at the BMx or LMx for a week, three biological replicates from MMECs and PDEC-N from both matrices and from the original samples immediately after isolation were directed to RNA sequencing. The sequencing confirmed that both matrices affected the expression profiles differently. Matrigel driven basal differentiation was evident from the transcriptomics data (FIG. 2C).

### Matrix Stiffness Regulates Nuclear Localization of Estrogen Receptor

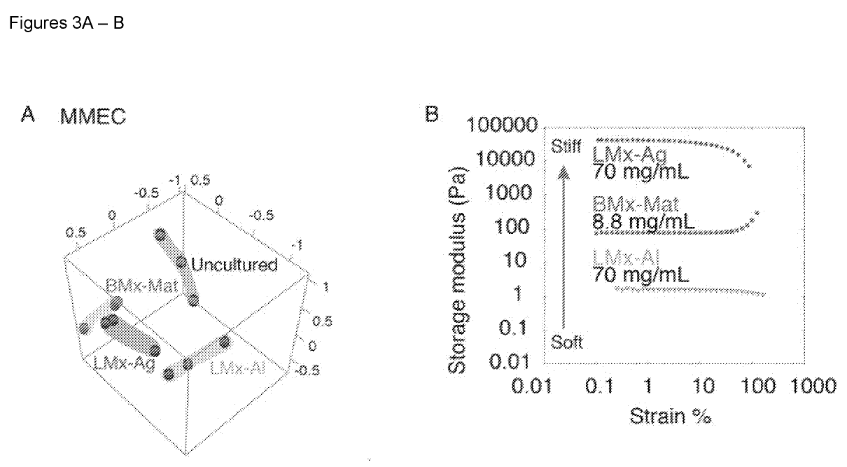

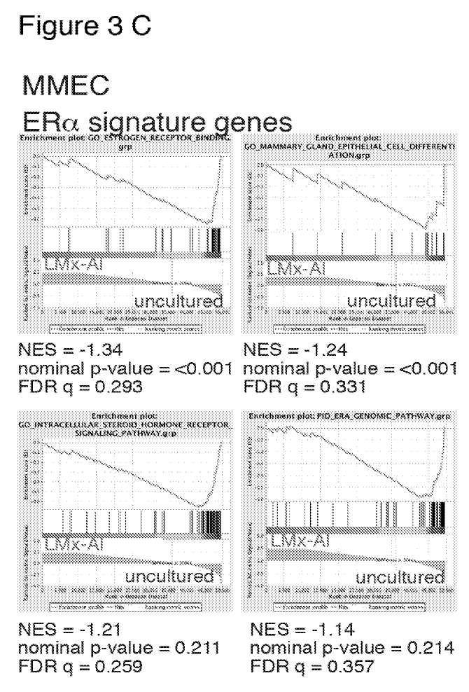

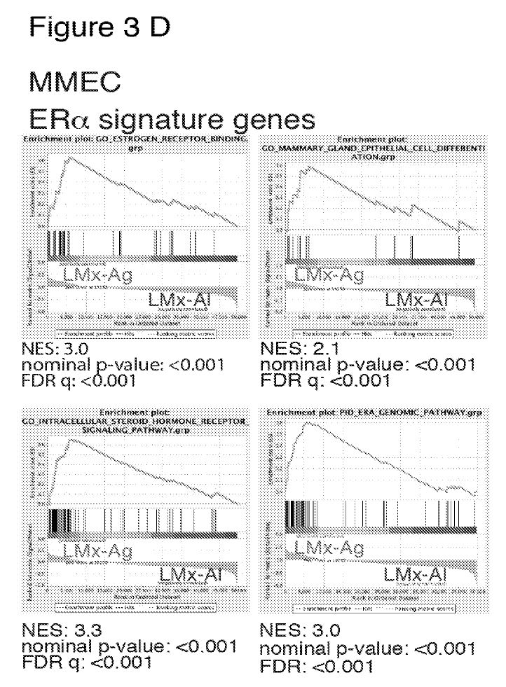

Our major finding suggests bioinert microenvironment to be the key determinant in nuclear estrogen receptor preservation. In addition to this, the transcription profiling with MMECs revealed a physical microenvironment as key regulator within luminal cell phenotype. Two bioinert hydrogels (LMx-Al and LMx-Ag) with different stiffness were included to RNA sequencing. Despite the bioinert nature of both matrices and that they both preserved the luminal cell identity, the samples clustered separately in PCA and indicated a difference in their gene expression profiles (FIG. 3A). To assess the differences in the physical stiffness, we measured the physical properties of different LMx matrices with rheology (FIG. 3B). The LMx-ag was several magnitudes stiffer than any other culture gel. Gene expression profiles indicating functional estrogen receptor signaling, accompanied by the nuclear localization of estrogen receptor alpha (ERα) were present only in LMx-Ag (FIG. 3C, D). We conclude that that sufficiently stiff microenvironment is required for nuclear ERα expression.

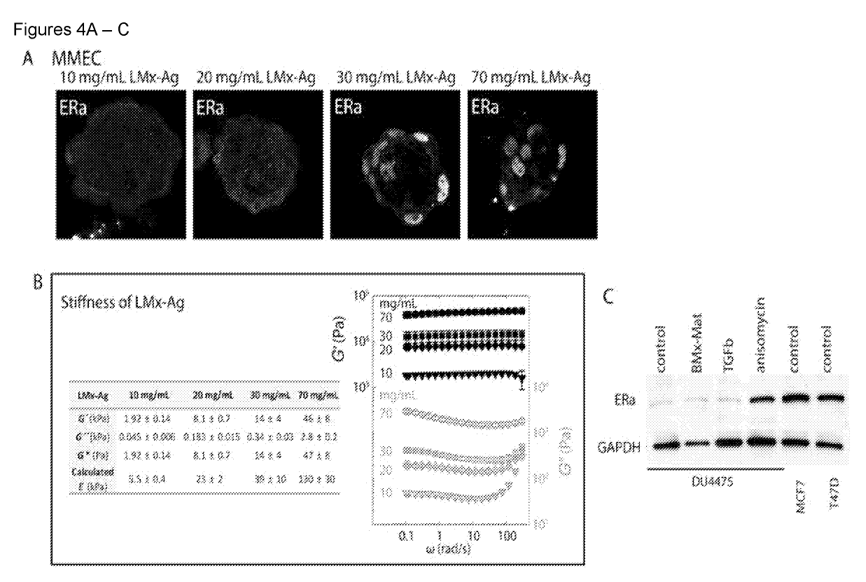

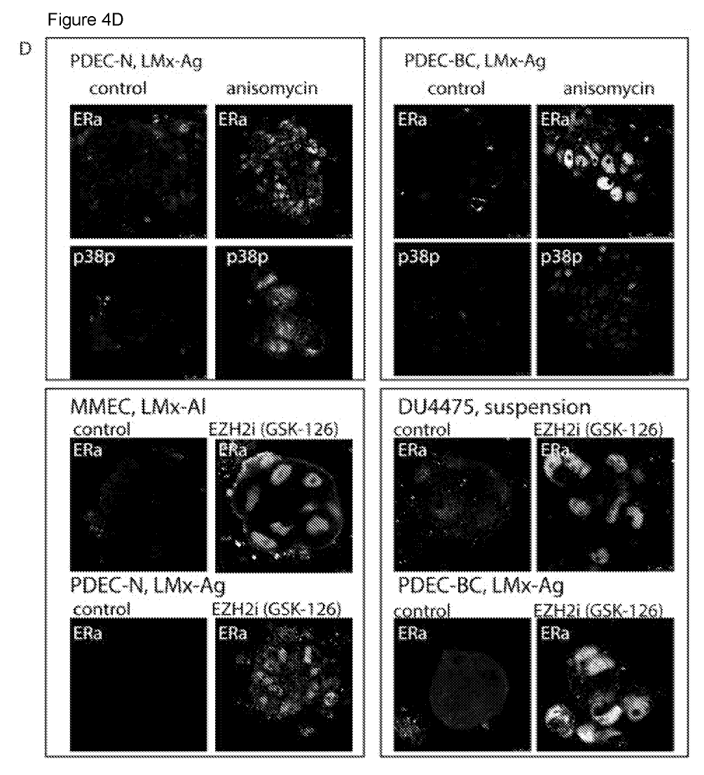

At least agarose matrix stiffness regulates nuclear localization of estrogen receptor The Ag stiffness, which induced the nuclear localization of ERα in MMECs, was determined with increasing polymer concentration (FIG. 4A). The corresponding increasing stiffness values were defined simultaneously with rheology (FIG. 4B). We can conclude that the nuclear localization of ERα in MMECs, was observed in sufficiently still matrices, i.e., beyond the Ag concentration of ca. 30 mg/mL hydrogel with at least 10 kPa stiffness presented as storage modulus measured using dynamic rheology (FIG. 4A and B).

### Tissue Stiffness Correlates With Higher ERα Expression in Vivo

Most solid tumors are several magnitudes stiffer than the normal tissues (Cell. 2009 Nov 25;139(5):891-906. doi: 10.1016/j.cell.2009.10.027.) In addition to this abnormal rigidity, the majority of breast cancer tissues are also ERα positive. We investigated, whether the ERα expression is an intrinsic feature of a cancer or more resulting from the mechanical properties of tumor tissue. We compared expression profiles of original tumor to equivalent tumor explants in LMx-Ag, which was able to maintain hormone receptor in MMECs. We observed, that the ERα regulated target genes were downregulated in 3D cultures, which correlated with the loss of nuclear ERα,suggesting that the LMx-Ag is too soft for human tumor tissue, when the culture is performed in a conventional way in a culture medium.

### Using Additional P38 Activating And/or EZH2 Inhibiting Compounds to Upregulate ERα

We observed stress-responsive mitogen-activated protein kinase (MAPKs) pathway activated in the in LMx-Ag, compared to LMx-Al. The nuclear expression of phosphorylated p38 was also visible in the MMECs cultured in LMx-Ag. To confirm the role of stress-responsive mitogen-activated protein kinases in the ERα activation, we used anisomycin to directly activate the pathway in TNBC cell line, DU4475. The treatment was able to activate the ERα expression in these cells (FIG. 4C). The activation of nuclear ERα was also observed in MMECS, PDEC-N and PDEC-T after anisomycin treatment. Nuclear localization of both ERα and phosphorylated-p38 was observed to colocalize in immunostained fragments (FIG. 4D). Our results showed that the loss of nuclear ERα in the soft 3D culture matrix is due to the repressive histone methylation, which takes place in the absence of stress. We used inhibitor of the methyltransferase EZH2, which mediates the silencing of ERα and observed the retained expression of ERα (FIG. 4D).

### Using Oxidative Stress to Upregulate ERα

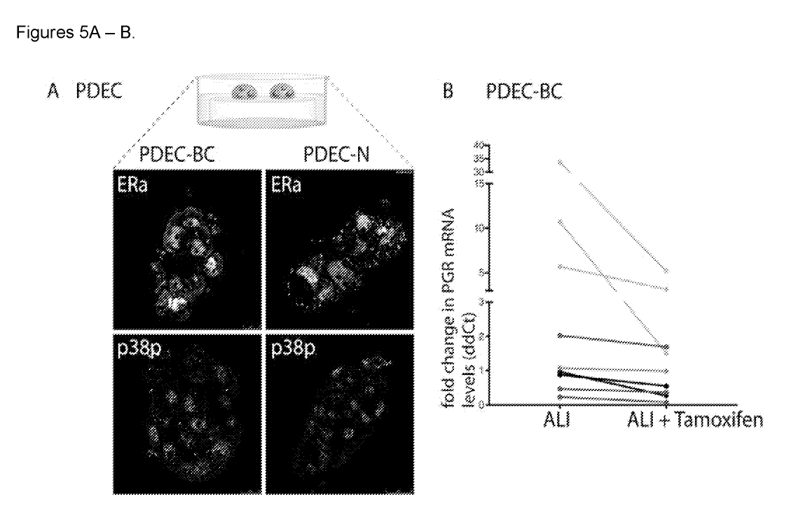

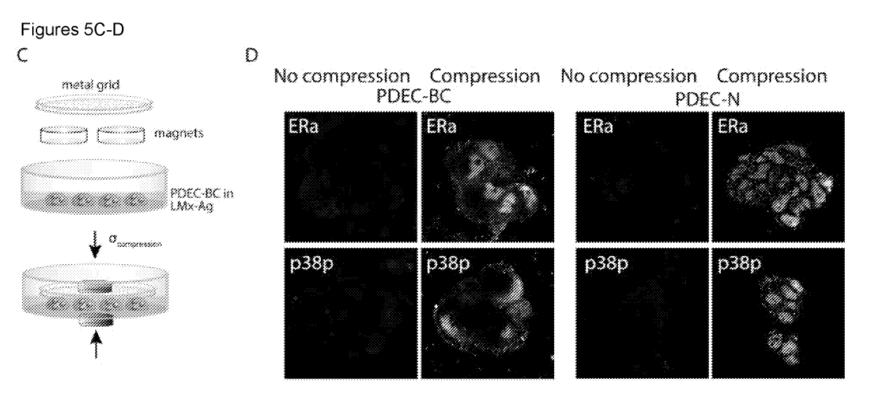

Instead of anisomycin, the stress activated protein kinase pathway is also activated by multiple other factors e.g oxidative stress. We observed, if the PDEC cultures were performed under a higher oxygen concentration, such as in air-liquid interface, the functional ERα was preserved together with the activation of p38 (FIG. 5A). We treated these PDEC-BC with antiestrogens and observed a suppression in ERα downstream targets PGR and GREB1 (FIG. 5B).

### Using Additional Compressive Forces to Upregulate ERα

The matrix stiffness of LMx-Ag was enough to upregulate the nuclear ERα expression in MMECs unlike in PDEC-N and PDEC-BC. We exposed the human PDEC-N and PDEC-BC cultures to the additional compressive forces with the magnet mediated mechanical compression (FIG. 5C). This was observed to upregulate ERα in human PDEC-N and PDEC-BC samples (FIG. 5D). We treated the compressed PDEC-BC with tamoxifen and observed a suppression in ERα downstream targets PGR and GREB1 (FIG. 5E).

### Collected Data of the Studied Matrices Obtained by Dynamic Rheology

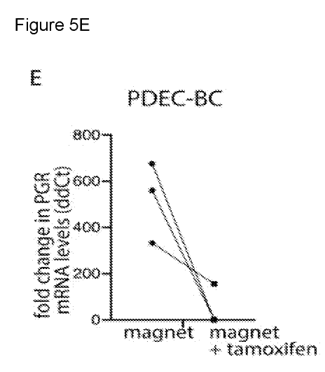

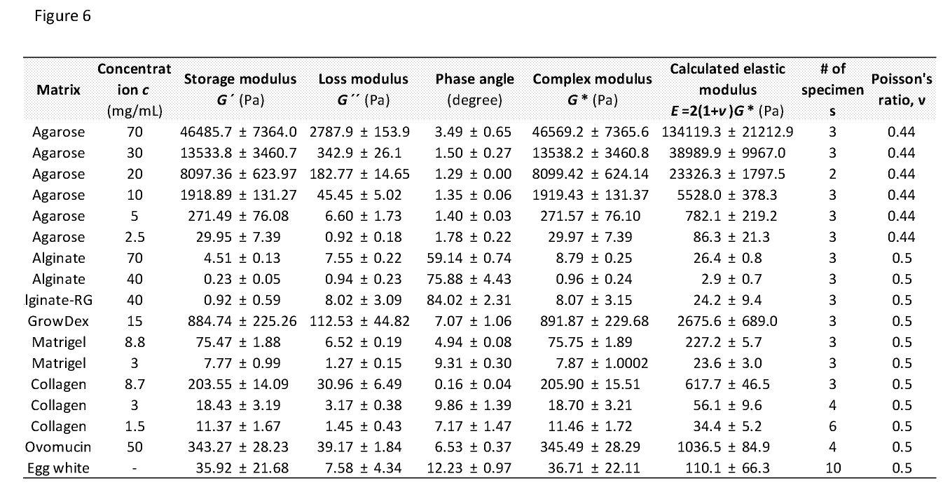

See FIG. 6 for the collected G′, G″, G* and E of the studied matrices (average values +/- standard deviation) measured by dynamic rheology.

1. A method of maintaining the presence or activity of a mammalian estrogen receptor (ER) in a primary cell of an ex vivo mammary cell or tissue culture or in a primary cell of other hormone responsive cell or tissue culture, wherein the method comprises culturing a mammalian primary mammary cell or tissue or other hormone responsive primary cell or tissue in a 3D matrix with storage modulus of at least about 10 kPa, optionally measured by dynamic rheology, or in a 3D medium comprising said matrix.

2. The method of claim 1, wherein the mammalian cell or tissue is selected from the group consisting of a human, rodent, mouse, murine, hamster, rabbit, swine, dog, and cat cell or tissue.

3. The method of claim 1, wherein the 3D matrix is substantially gelled, and/or the 3D medium and/or matrix is bioinert.

4. (canceled)

5. (canceled)

6. The method of any of claim 1, wherein the culture is exposed to an external stress or compound.

7. The method of claim 1,wherein the cell or tissue is cultured in the 3D matrix or 3D medium in presence of a stress pathway inducing compound or method, in the presence of a culture medium comprising anisomycin, in presence of an epigenetic pathway modulating compound that optionally influences H3K27 methylation, in presence of an inhibitor of the methyltransferase EZH2, or in presence of a culture medium comprising an inhibitor of the methyltransferase EZH2 .

8. (canceled)

9. (canceled)

10. The method of claim 1,wherein storage modulus of the 3D matrix under suitable culture conditions is achieved by using external mechanical compression or magnetic compression.

11. The method of claim 1,wherein the 3D matrix comprises alginate, agarose preferably at a concentration above about 20 mg/mL, ovomucin, egg white or any combination thereof.

12. (canceled)

13. The method of claim 1,wherein the cell is a tumor cell, non-tumor cell or a cell from a cell line, or the tissue comprises tumor cells and/or non-tumor cells.

14. The method of any of claim 1 , wherein the cell of other hormone responsive cell is selected from the group consisting of a cell of the prostate, chondrocyte, kidney cell, pancreatic cell, bladder cell and ovarian cell, or the other hormone responsive tissue comprises cells selected from the group consisting of cells of the prostate, chondrocytes, kidney cells, pancreatic cells, bladder cells and ovarian cells.

15. A method of maintaining a luminal epithelial phenotype and/or luminal epithelial cell identity of a mammalian primary cell in an ex vivo cell or tissue culture, wherein the method comprises culturing a mammalian primary cell in a 3D matrix with storage modulus of at least about 10 kPa, optionally measured by dynamic rheology, or in a 3D medium comprising said matrix.

16. The method of claim 15, wherein the 3D matrix is substantially gelled, and/or the 3D medium and/or matrix is bioinert.

17. The method of claim 15 , wherein the 3D matrix comprises alginate, agarose preferably at a concentration above about 20 mg/mL, ovomucin, egg white or any combination thereof.

18. (canceled)

19. The method of claim 15, wherein the cell is a tumor cell, non-tumor cell or a cell from a cell line, or the tissue comprises tumor cells and/or non-tumor cells.

20. (canceled)

21. The method of claim 15 , wherein the luminal epithelial phenotype or luminal epithelial cell identity of the cell is characterized or determined by
the presence of at least one or more cytokeratins optionally selected from the group consisting of notch, cytokeratin 8 (CK8) and cytokeratin 18 (CK18),
the lack of one or more basal cytokeratins optionally selected from the group consisting of ΔNp63, cytokeratin 5 (CK5) and cytokeratin 14 (CK14),
a gene expression profile associated with a luminal phenotype, and/or the lack of a gene expression profile associated with a basal phenotype.

22. A 3D matrix or 3D medium comprising the matrix for ex vivo culture, wherein said 3D matrix or 3D medium comprises one or more mammalian primary cells or primary tissues embedded in said 3D matrix or 3D medium, wherein storage modulus of the matrix is at least about 10 kPa, optionally measured by dynamic rheology, and the 3D matrix or 3D medium is capable of maintaining the presence or activity of an estrogen receptor (ER), a luminal epithelial phenotype and/or cell identity of the cell or tissue embedded in said matrix or 3D medium.

23. A system for ex vivo culture, wherein the system comprises one or more mammalian primary cells or primary tissues embedded in a 3D matrix or 3D medium comprising said matrix, wherein storage modulus of the 3D matrix is at least about 10 kPa, optionally measured by dynamic rheology, and the 3D matrix or 3D medium is capable of maintaining the presence or activity of an estrogen receptor (ER), a luminal epithelial phenotype and/or cell identity of the cell or tissue embedded in said matrix or 3D medium.

24. The 3D matrix, or 3D medium of claim 22, wherein the 3D matrix or 3D medium is substantially gelled or comprises a substantially gelled material, and/or the 3D matrix or 3D medium is bioinert.

25. The 3D matrix, or 3D medium of claim 22, wherein the 3D matrix comprises alginate, agarose preferably at a concentration above about 20 mg/mL, ovomucin, egg white or any combination thereof.

26. (canceled)

27. The 3D matrix, or 3D medium of claim 22, wherein the cell is a tumor cell, non-tumor cell or a cell from a cell line, or the tissue comprises tumor cells and/or non-tumor cells.

28. (canceled)

29. (canceled)

30. (canceled)

31. The system of claim 23, wherein the 3D matrix or 3D medium is substantially gelled or comprises a substantially gelled material, and/or the 3D matrix or 3D medium is bioinert.

32. The system of claim 23, wherein the 3D matrix comprises alginate, agarose preferably at a concentration above about 20 mg/mL, ovomucin, egg white or any combination thereof.

33. The system of claim 23, wherein the cell is a tumor cell, non-tumor cell or a cell from a cell line, or the tissue comprises tumor cells and/or non-tumor cells.
